(19) United States
(12) Patent Application Publication
(10) Pub. No.: US 2024/0392309 A1
(43) Pub. Date: Nov. 28, 2024

(54) A TRANSGENIC SOYBEAN EVENT CAL16 AND ITS DETECTION METHOD

(71) Applicant: HANGZHOU RUIFENG BIOSCIENCE CO., LTD., Hangzhou (CN)

(72) Inventors: Ting ZHENG, Hangzhou (CN); Pengfei WANG, Hangzhou (CN); Chao XU, Hangzhou (CN); Haiyan LIN, Hangzhou (CN); Yuanyuan JIANG, Hangzhou (CN); Xuezhen XU, Hangzhou (CN); Mengzhen TANG, Hangzhou (CN); Jing LI, Hangzhou (CN); Hongying TONG, Hangzhou (CN); Hong JIANG, Hangzhou (CN); Chun JIANG, Hangzhou (CN); Chaoyang LIN, Hangzhou (CN); Zhicheng SHEN, Hangzhou (CN)

(21) Appl. No.: 18/696,865

(22) PCT Filed: Feb. 2, 2023
(86) PCT No.: PCT/CN2023/074171

§ 371 Date: Mar. 28, 2024

(30) Foreign Application Priority Data
Sep. 7, 2022 (CN) 202211088062.3

(51) Int. Cl. C12N 15/82; C12Q 1/6895

(52) U.S. Cl. CPC C12N 15/8275; C12N 15/8286; C12Q 1/6895; C12Q 2600/13; C12Q 2600/156

(57) ABSTRACT

The present invention relates to a transgenic soybean event CAL16 and its detection method thereof. The transgenic soybean event CAL16 is obtained by inserting an exogenous gene (i.e., T-DNA) between the 3′ end shown in SEQ ID NO:27 and the 5′ end shown in SEQ ID NO:28 of the soybean genome chromosome 18. The transgenic soybean plant CAL16 of the present invention exhibits good resistance to lepidopteran insects and good tolerance to the herbicide glyphosate, without affecting yield. Furthermore, the detection method enables accurate and rapid identification of whether a biological sample contains the DNA molecule of the transgenic soybean event CAL16.

### REFERENCE TO AN ELECTRONIC SEQUENCE LISTING

The content of the electronic sequence listing (2024-03-28-Sequence-Listing.xml; Size: 47 KB; and Date of Creation: Mar. 28, 2024) is herein incorporated by reference in its entirety.

### TECHNICAL FIELD

The present invention relates to the transgenic soybean event and its identification method. Specifically, it involves the insertion of exogenous genes into the soybean cell genome to create the transgenic soybean event CAL16. It also includes specific primers, probes, and methods for detecting this event. The invention further encompasses microorganisms and products derived from the transgenic soybean event CAL16. The exogenous genes in this event comprise a fused gene encoding insecticidal protein and a glyphosate tolerance gene.

### BACKGROUND ART

Soybean (Glycine max) is an important crop in many regions of the world, and biotechnological methods have been applied to develop soybean varieties with desired traits. The two most important desired traits are insect resistance and herbicide tolerance. Expression of insect resistance and herbicide tolerance transgenes in plants can confer the desired traits to the plants. However, the expression of transgenes is influenced by various factors, including the orientation and composition of the gene expression cassette driving the target gene into the plant chromosome, the chromosomal location, and the genomic outcome of the transgene insertion. For example, variations have been observed in the levels and patterns of transgene expression in individual events with different chromosomal insertion sites but otherwise similar characteristics in plants. Undesired and/or desired phenotypic or agronomic differences also exist among different events. Therefore, it is often necessary to generate and analyze a large number of individual plant cell transformation events to select events that possess the desired traits, optimal phenotypes, and agricultural characteristics suitable for commercial success. Selecting preferred transgenic events requires extensive molecular characterization and greenhouse and field trials of numerous events over many years, in multiple locations, and under various conditions. Significant amounts of efficacy, phenotypic, and molecular data are collected, and the resulting data and observations are analyzed by teams of scientists and agronomists with the goal of selecting one or more commercially viable events. Subsequently, the selected events with the desired transgenic traits are incorporated into other genetic backgrounds using plant breeding methods, resulting in the development of numerous different crop varieties that possess the desired traits and are appropriately suited for specific local agronomic conditions. Currently, this method is time-consuming and labor-intensive. Moreover, trait segregation occurs in the hybrid progeny, rendering them unsuitable for seed retention, and the breeding process is slow and complex. The outcome of hybridization is unpredictable, requiring extensive selection and seed production efforts, with poor performance in subsequent generations.

In addition, transgenic soy beans that rely on the expression of a single toxin for insect pest control may be at risk of limited durability, as there is an increased possibility of insect pests developing resistance to the toxin. It is beneficial to have soybean plants that express multiple toxins simultaneously to manage resistance risks effectively, compared to soybeans expressing a single toxin. Soybean transformation events containing three different lepidopteran insect resistance traits have been publicly disclosed. These include events expressing the Cry1Ac toxin protein, events expressing both Cry1Ac and Cry1F toxin proteins, and events expressing Cry1A.105 and Cry2Ab in soybeans. This represents a shift in strategy from introducing a single toxin gene to introducing multiple toxin genes. The simultaneous introduction of insect resistance genes and herbicide resistance genes into soybean crops is an important development trend in transgenic soybeans.

It is known that the expression of exogenous genes in plants is influenced by their chromosomal location, possibly due to chromatin structure (such as heterochromatin) or the proximity of transcriptional regulatory elements (such as enhancers) to the integration site. Therefore, it is often necessary to screen a large number of events to identify those that can be commercialized (i.e., events where the target gene is optimally expressed upon integration). For example, significant differences in gene expression levels have been observed among different events in plants and other organisms. Differences may also exist in the spatial or temporal patterns of expression, such as variations in relative transgene expression among different plant tissues. These differences may manifest as actual expression patterns that are inconsistent with the expected expression patterns based on the transcriptional regulatory elements present in the constructed transgene cassette. Therefore, it is typically necessary to generate hundreds or thousands of different events and select a single event that exhibits the desired transgene expression levels and patterns for commercialization purposes. Once an event with the expected transgene expression levels and patterns is obtained, it can be further incorporated into other genetic backgrounds using conventional breeding methods through sexual outcrossing. The progeny produced through this hybridization method maintain the transgene expression characteristics of the original transformation event.

It would be beneficial to have methods for detecting the presence of specific events to determine if the progeny of sexual crosses contain the desired genes. Additionally, methods for detecting specific events would aid in compliance with relevant regulations, such as the requirement for formal approval and labeling of food derived from genetically engineered crops before they are introduced into the market. It is possible to detect the presence of transgenes using any well-known nucleic acid detection methods, such as polymerase chain reaction (PCR) or DNA hybridization using nucleic acid probes. These detection methods typically target commonly used genetic elements, such as promoters, terminators, and marker genes. Therefore, unless the sequence of the chromosomal DNA (“flanking DNA”) adjacent to the inserted transgenic DNA is known, the aforementioned methods cannot distinguish between different events, especially those generated using the same DNA construct.

### SUMMARY OF THE INVENTION

The purpose of this invention is to provide a transgenic soybean event CAL16, and its detection method. The invention involves the insertion of exogenous genes into a specific site of the soybean cell genome to construct the transgenic soybean event CAL16. The insertion site of the exogenous genes is clearly defined, addressing the problem of the existing methods' inability to accurately and rapidly identify biological samples. The invention utilizes a pair of primers that span the junction between the inserted exogenous genes and the flanking DNA of the soybean genome for the identification of the specific transgenic event using PCR. Specifically, the first primer contains the flanking sequence, while the second primer contains the inserted sequence, overcoming the limitations of existing methods in distinguishing between different events. With the nucleotide sequences, primers, probes, and detection methods provided by this invention, the presence of the DNA molecule of the transgenic soybean event CAL16 in biological samples can be accurately and rapidly identified. The exogenous genes in this invention can be any genes, such as including an insect resistance gene expression cassette and a glyphosate tolerance gene expression cassette. The insect resistance gene expression cassette overcomes the problem of insect resistance in transgenic events by expressing a fusion protein, Cry1Ab/Vip3Da, toxic to lepidopteran insect species, especially reducing resistance in lepidopteran pests such as fall armyworm, beet armyworm, cotton bollworm, and soybean looper. Simultaneously, the glyphosate tolerance gene expression cassette encodes G10evo EPSPS, providing soybean plants with tolerance to glyphosate herbicide.

To achieve the purpose mentioned above, the technical solution employed by the present invention is:

The first aspect of the present invention provides a transgenic soybean event, CAL16, which is obtained by inserting exogenous genes (T-DNA) into the soybean genome between the 3′ end as shown by SEQ ID NO:27 and the 5′ end as shown by SEQ ID NO:28. The nucleotide sequence of the soybean genome is sourced from NCBI: RefSeq Genome Database (Glycine max cultivar Williams 28-v4.0). The exogenous genes include a glyphosate tolerance gene expression cassette and an insect resistance gene expression cassette. The glyphosate tolerance gene expression cassette comprises the following components: a CaMV 35S promoter used for the expression of the glyphosate tolerance gene g10evo-epsps, an Arabidopsis EPSPS chloroplast signal peptide, the g10evo-epsps gene, and the CaMV 35S terminator. The insect resistance gene expression cassette comprises a pCsVMV promoter, the cry1Ab/vip3Da fusion gene for insect resistance, and the NOS terminator.

SEQ ID NO. 27
TTTGGCAAAGGGAGAAGGGAATGAAAAAGATGAATAGCACAAGTTTTCA
AGGTTTGGAAAAACCAAAAAACTTTGGAAAGCTTTTAGCAAAAGGAAGG
AGAAGAAGAAGAAGAAGAAGAAGAAGTTCAAAGAGATTCAAGGCTTGTA
AAGGATTGTATAAGATTGATTGGAAAAGTGTATTGAAAAGCAAATCATG
TCAACATTAATGAGCCTGGGATGCTCTAAACACATGATTGGAGATGCAT
CAAAGCTCACTCATATTTCTCCCAAGAATAGTGGACATGTGACTTATGG
CGACACCAATAATGGTAGAATCCTTGGAGTTGGAAAAACTCTTGTGTGT
TTGTCTCCTTCTTCCCTACTCTTTTACTTTCCATTGTGCATTTTAATTT
CCTCTTTTACTTTCTGTTAAGTTTCTCTTCTACTCCTCATGTCAACATA
TC.
SEQ ID NO. 28
CACTATTATTGTTTTTGTATTATATTTTCCCAGGCTTATCTTTTATCAA
TGAGTTATATATAAAGAAATAATCAGTCAACATGTAGCAACAAAATATT
TGCAGTAATAATAATAACGTTAAACAATAGAAATTAAAAAAACCTAACA
ACAAATGTCTTGATTTTAAAGACTTGTGTTCACATGATCATTTGATCAA
GTAAAAGATAACTTCCTAAGGCAAGAATGAAAGTAGTGAGATGTTTTTA
GTTTTTTTTTTCCACACAAACTAATTCTAAAAACATTTTATCTTAAACA
AAAATACAATTAACAAAGAAAATATTTTTCAACAATCTCATACTAATGT
AAAACTTCAGGAAATACATAATATAATAAAAACATTTTTAATGGAACAT
CATGCATTGTGTTTTCATCCATTAATTTTTCATGCTTACTAGAAAGGAA
CTTAATCATATCCATTATAGATATTTTGATACAACATCATGCTACTGCA
GAAAAGACTATGCAAGAAGAAAGTATAAAACATTTTTCTCTCTTAAGAC
TGTTGGAAAATAAAACAAAAATGAAGGAAAATAAATACGAAGAAGATGC
ACAGTCTTGAATTA.

Preferably, the nucleotide sequence of the DNA molecule of the transgenic soybean event CAL16 is shown as SEQ ID NO:10.

Preferably, the transgenic soybean event CAL16 is preserved in the form of seeds at the China Center for Type Culture Collection (CCTCC), with the accession number CCTCC NO: P202205. The preservation date is Apr. 18, 2022, and the address is Wuhan University, Wuhan, China, postal code 430072.

It is worth noting that researchers in the field are well aware that the soybean genome contains a significant number of active transposon sequences, and there may be positional variations of sequences in the soybean genome among different genetic backgrounds. Researchers in the field can obtain progeny of the transgenic soybean event CAL16 through methods such as hybridization. Any soybean event in the progeny whose flanking sequences of the exogenous T-DNA match SEQ ID NO:27 and SEQ ID NO:28 should be considered within the scope of the present invention.

The second aspect of the present invention provides a nucleotide sequence for detecting the transgenic soybean event CAL16. The nucleotide sequence includes SEQ ID NO:1 or its complementary sequence, and/or SEQ ID NO:2 or its complementary sequence.

SEQ ID NO: 1:
tcaacatatctcaaacactg atagt;
SEQ ID NO: 2:
ttaagttgtccactattattgtttt.

The SEQ ID NO:1 or its complementary sequence is a 25-nucleotide sequence located near the 5′ end of the inserted sequence at the junction site in the transgenic soybean event CAL16. The SEQ ID NO:1 or its complementary sequence spans the flanking genomic DNA sequence of the soybean insertion site and the 5′ end of the inserted sequence. The presence of SEQ ID NO:1 or its complementary sequence can be used to identify the existence of the transgenic soybean event CAL16. The SEQ ID NO:2 or its complementary sequence is a 25-nucleotide sequence located near the 3′ end of the inserted sequence at the junction site in the transgenic soybean event CAL16. The SEQ ID NO:2 or its complementary sequence spans the 3′ end of the inserted sequence and the flanking genomic DNA sequence of the soybean insertion site. The presence of SEQ ID NO:2 or its complementary sequence can be used to identify the existence of the transgenic soybean event CAL16.

Furthermore, the nucleotide sequence of the present invention also includes SEQ ID NO:3 or its complementary sequence, and/or SEQ ID NO:4 or its complementary sequence.

SEQ ID NO: 3:
aagtttctct tctactcctc atgtcaacat atctcaaaca
ctgatagttt aaactgaagg;
SEQ ID NO: 4:
acgtccgcaa tgtgttatta agttgtccac tattattgtt
tttgtattat attttcccag.

The SEQ ID NO:3 or its complementary sequence is a 60-nucleotide sequence located near the 5′ end of the inserted sequence at the junction site in the transgenic soybean event CAL16. The SEQ ID NO:3 or its complementary sequence spans the flanking genomic DNA sequence of the soybean insertion site and the 5′ end of the inserted sequence. The presence of SEQ ID NO:3 or its complementary sequence can be used to identify the existence of the transgenic soybean event CAL16. The SEQ ID NO:4 or its complementary sequence is a 60-nucleotide sequence located near the 3′ end of the inserted sequence at the junction site in the transgenic soybean event CAL16. The SEQ ID NO:4 or its complementary sequence spans the 3′ end of the inserted sequence and the flanking genomic DNA sequence of the soybean insertion site. The presence of SEQ ID NO:4 or its complementary sequence can be used to identify the existence of the transgenic soybean event CAL16.

Furthermore, the nucleotide sequence of the present invention also includes SEQ ID NO:5 or its complementary sequence, and/or SEQ ID NO:6 or its complementary sequence.

SEQ ID NO: 5:
ttcctctttt actttctgtt aagtttctct tctactcctc
atgtcaacat atctcaaaca ctgatagttt aaactgaagg
cgggaaacga caatctgatc;
SEQ ID NO: 6:
gttaattcag tacattaaaa acgtccgcaa tgtgttatta
agttgtccac tattattgtt tttgtattat attttcccag
gcttatcttt tatcaatgag.

The SEQ ID NO:5 or its complementary sequence is a 100-nucleotide sequence located near the 5′ end of the inserted sequence at the junction site in the transgenic soybean event CAL16. The SEQ ID NO:5 or its complementary sequence spans the flanking genomic DNA sequence of the soybean insertion site and the 5′ end of the inserted sequence. The presence of SEQ ID NO:5 or its complementary sequence can be used to identify the existence of the transgenic soybean event CAL16. The SEQ ID NO:6 or its complementary sequence is a 100-nucleotide sequence located near the 3′ end of the inserted sequence at the junction site in the transgenic soybean event CAL16. The SEQ ID NO:6 or its complementary sequence spans the 3′ end of the inserted sequence and the flanking genomic DNA sequence of the soybean insertion site. The presence of SEQ ID NO:6 or its complementary sequence can be used to identify the existence of the transgenic soybean event CAL16.

Furthermore, the nucleotide sequence of the present invention also includes SEQ ID NO:7 or its complementary sequence, and/or SEQ ID NO:8 or its complementary sequence.

SEQ ID NO: 7:
tttggcaaag ggagaaggga atgaaaaaga tgaatagcac aagttttcaa ggtttggaaa
aaccaaaaaa ctttggaaag cttttagcaa aaggaaggag aagaagaaga agaagaagaa
gaagttcaaa gagattcaag gcttgtaaag gattgtataa gattgattgg aaaagtgtat
tgaaaagcaa atcatgtcaa cattaatgag cctgggatgc tctaaacaca tgattggaga
tgcatcaaag ctcactcata tttctcccaa gaatagtgga catgtgactt atggcgacac
caataatggt agaatccttg gagttggaaa aactcttgtg tgtttgtctc cttcttccct
actcttttac tttccattgt gcattttaat ttcctctttt actttctgtt aagtttctct
tctactcctc atgtcaacat atctcaaaca ctgatagttt aaactgaagg cgggaaacga
caatctgatc caagctcaag ctgctctagc attcgccatt caggctgcgc aactgttggg
aagggcgatc ggtgcgggcc tcttcgctat tacgccagct ggcgaaaggg ggatgtgctg
caaggcgatt aagttgggta acgccagggt tttcccagtc acgacgttgt aaaacgacgg
ccagtgccaa gcttccagaa ggtaattatc caagatgtag catcaagaat ccaatgttta
cgggaaaaac tatggaagta ttatgtgaac tcagcaagaa gcagatcaat atgcggcaca
tatgcaacct atgttcaaaa atgaagaatg tacagataca agatcctata ctgccagaat
acgaagaaga atacgtagaa attgaaaaag aagaaccagg cgaagaaaag aatcttgaag
acgtaagcac tgacgacaac aatgaaaaga agaagataag gtcggtgatt gtgaaagaga
catagaggac acatgtaagg tggaaaatgt aagggcggaa agtaacctta tcacaaagga
atcttatccc ccactactta tccttttata tttttccgtg tcactagtga agacgtaagc
actgacgaca acaatgaaaa gaagaagata aggtcggtga ttgtgaaaga gacatagagg
acacatgtaa ggtggaaaat gtaagggcgg aaagtaacct tatcacaaag gaatcttatc
ccccactact tatcctttta tatttttccg tgtcattttt gcccttgagt tttcctatat
aaggaaccaa gttcggcatt tgtgaaaaca agaaaaaatt tggtgtaagc tattttcttt
gaagtactga ggatacaact tcagagaaat ttgtaagttt gtggatccaa caatggacaa
caaccccaac atcaacgagt gcatccccta caactgcctg agcaaccccg aggtggaggt
gctgggcggc gagcgcatcg agaccggcta cacccccatc gacatcagcc tgagcctgac
ccagttcctg ctgagcgagt tcgtgcccgg cgccggcttc gtgctgggcc tggtggacat
catctggggc atcttcggcc ccagccagtg ggacgccttc ctggtgcaga.
SEQ ID NO: 8:
atgttcctcg gtcctcttct tcctgacgga ctcgaactta gactcaccgg tgatatcaag
tcccacgctc ctcttagaca gacacttgac accctctctg atttcggtgt tagagctact
gcctccgatg accttagaag aatctccatc cctggtggtc agaagtacag accaggtaga
gtgctcgttc ctggtgatta ccctggttcc gctgctatcc ttaccgccgc tgctcttctc
ccaggtgagg ttagactttc taaccttaga gaacacgacc tccagggtga gaaggaagct
gtgaacgttc ttagagagat gggtgctgat atcgttagag aaggtgatac ccttaccgtg
agaggtggta gacctctcca cgctgttact agagatggtg attccttcac cgacgccgtg
caagctctta ccgctgctgc tgccttcgct gagggtgata ccacctggga aaacgttgct
actcttagac tcaaggaatg cgatagaatc tctgacacca gagctgagct tgaaagactt
ggtcttagag caagagagac cgccgattct ctctccgtta ctggttctgc tcaccttgct
ggtggtatca ccgctgatgg tcacggtgac cacagaatga tcatgcttct cacccttctt
ggtctcagag cagatgctcc acttagaatc accggtgcac accacatcag aaagtcctac
cctcagttct tcgctcacct tgaagctctt ggtgctagat tcgaatacgc tgaggctacc
gcctaatagg agctcgagtt tctccataat aatgtgtgag tagttcccag ataagggaat
tagggttcct atagggtttc gctcatgtgt tgagcatata agaaaccctt agtatgtatt
tgtatttgta aaatacttct atcaataaaa tttctaattc ctaaaaccaa aatccagtac
taaaatccag atcccccgaa ttaattcggc gttaatccag tacattaaaa acgtccgcaa
tgtgttatta agttgtccac tattattgtt tttgtattat attttcccag gcttatcttt
tatcaatgag ttatatataa agaaataatc agtcaacatg tagcaacaaa atatttgcag
taataataat aacgttaaac aatagaaatt aaaaaaacct aacaacaaat gtcttgattt
taaagacttg tgttcacatg atcatttgat caagtaaaag ataacttcct aaggcaagaa
tgaaagtagt gagatgtttt tagttttttt tttccacaca aactaattct aaaaacattt
tatcttaaac aaaaatacaa ttaacaaaga aaatattttt caacaatctc atactaatgt
aaaacttcag gaaatacata atataataaa aacattttta atggaacatc atgcattgtg
ttttcatcca ttaatttttc atgcttacta gaaaggaact taatcatatc cattatagat
attttgatac aacatcatgc taccgcagaa aagactatgc aagaagaaag tataaaacat
ttttctctct taagactgtt ggaaaataaa acaaaaatga aggaaaataa atacgaagaa
gatgcacagt cttgaatta.

The SEQ ID NO:7 or its complementary sequence is a 1610-nucleotide sequence located near the 5′ end of the inserted sequence at the junction site in the transgenic soybean event CAL16. The SEQ ID NO:7 or its complementary sequence consists of a 443-nucleotide soybean flanking genomic DNA sequence (nucleotides 1-443 of SEQ ID NO:7) and a 1167-nucleotide pCAL construct DNA sequence (nucleotides 444-1610 of SEQ ID NO:7). The presence of SEQ ID NO:7 or its complementary sequence can be used to identify the existence of the transgenic soybean event CAL16. The SEQ ID NO:8 or its complementary sequence is a 1639-nucleotide sequence located near the 3′ end of the inserted sequence at the junction site in the transgenic soybean event CAL16. The SEQ ID NO:8 or its complementary sequence consists of a 1037-nucleotide pCAL construct DNA sequence (nucleotides 1-1037 of SEQ ID NO:8) and a 602-nucleotide soybean integration site flanking genomic DNA sequence (nucleotides 1038-1639 of SEQ ID NO:8). The presence of SEQ ID NO:8 or its complementary sequence can be used to identify the existence of the transgenic soybean event CAL16.

Furthermore, the nucleotide sequence includes SEQ ID NO:10 or its complementary sequence.

tttggcaaag ggagaaggga atgaaaaaga tgaatagcac aagttttcaa ggtttggaaa
aaccaaaaaa ctttggaaag cttttagcaa aaggaaggag aagaagaaga agaagaagaa
gaagttcaaa gagattcaag gcttgtaaag gattgtataa gattgattgg aaaagtgtat
tgaaaagcaa atcatgtcaa cattaatgag cctgggatgc tctaaacaca tgattggaga
tgcatcaaag ctcactcata tttctcccaa gaatagtgga catgtgactt atggcgacac
caataatggt agaatccttg gagttggaaa aactcttgtg tgtttgtctc cttcttccct
actcttttac tttccattgt gcattttaat ttcctctttt actttctgtt aagtttctct
tctactcctc atgtcaacat atctcaaaca ctgatagttt aaactgaagg cgggaaacga
caatctgatc caagctcaag ctgctctagc attcgccatt caggctgcgc aactgttggg
aagggcgatc ggtgcgggcc tcttcgctat tacgccagct ggcgaaaggg ggatgtgctg
caaggcgatt aagttgggta acgccagggt tttcccagtc acgacgttgt aaaacgacgg
ccagtgccaa gcttccagaa ggtaattatc caagatgtag catcaagaat ccaatgttta
cgggaaaaac tatggaagta ttatgtgaac tcagcaagaa gcagatcaat atgcggcaca
tatgcaacct atgttcaaaa atgaagaatg tacagataca agatcctata ctgccagaat
acgaagaaga atacgtagaa attgaaaaag aagaaccagg cgaagaaaag aatcttgaag
acgtaagcac tgacgacaac aatgaaaaga agaagataag gtcggtgatt gtgaaagaga
catagaggac acatgtaagg tggaaaatgt aagggcggaa agtaacctta tcacaaagga
atcttatccc ccactactta tccttttata tttttccgtg tcactagtga agacgtaagc
actgacgaca acaatgaaaa gaagaagata aggtcggtga ttgtgaaaga gacatagagg
acacatgtaa ggtggaaaat gtaagggcgg aaagtaacct tatcacaaag gaatcttatc
ccccactact tatcctttta tatttttccg tgtcattttt gcccttgagt tttcctatat
aaggaaccaa gttcggcatt tgtgaaaaca agaaaaaatt tggtgtaagc tattttcttt
gaagtactga ggatacaact tcagagaaat ttgtaagttt gtggatccaa caatggacaa
caaccccaac atcaacgagt gcatccccta caactgcctg agcaaccccg aggtggaggt
gctgggcggc gagcgcatcg agaccggcta cacccccatc gacatcagcc tgagcctgac
ccagttcctg ctgagcgagt tcgtgcccgg cgccggcttc gtgctgggcc tggtggacat
catctggggc atcttcggcc ccagccagtg ggacgccttc ctggtgcaga tcgagcagct
gatcaaccag cgcatcgagg agttcgcccg caaccaggcc atcagccgcc tggagggcct
gagcaacctg taccaaatct acgccgagag cttccgcgag tgggaggccg accccaccaa
ccccgccctg cgcgaggaga tgcgcatcca gttcaacgac atgaacagcg ccctgaccac
cgccatcccc ctgttcgccg tgcagaacta ccaggtgccc ctgctgagcg tgtacgtgca
ggccgccaac ctgcacctga gcgtgctgcg cgacgtcagc gtgttcggcc agcgctgggg
cttcgacgcc gccaccatca acagccgcta caacgacctg acccgcctga tcggcaacta
caccgaccac gccgtgcgct ggtacaacac cggcctggag cgcgtgtggg gtcccgacag
ccgcgactgg atcaggtaca accagttccg ccgcgagctg accctgaccg tgctggacat
cgtgagcctg ttccccaact acgacagccg cacctacccc atccgcaccg tgagccagct
gacccgcgag atttacacca accccgtgct ggagaacttc gacggcagct tccgcggcag
cgcccagggc atcgagggca gcatccgcag cccccacctg atggacatcc tgaacagcat
caccatctac accgacgccc accgcggcga gtactactgg agcggccacc agatcatggc
cagccccgtc ggcttcagcg gccccgagtt caccttcccc ctgtacggca ccatgggcaa
cgctgcacct cagcagcgca tagtggcaca gctgggccag ggagtgtacc gcaccctgag
cagcaccctg taccgtcgac ctttcaacat cggcatcaac aaccagcagc tgagcgtgct
ggacggcacc gagttcgcct acggcaccag cagcaacctg cccagcgccg tgtaccgcaa
gagcggcacc gtggacagcc tggacgagat cccccctcag aacaacaacg tgccacctcg
acagggcttc agccaccgtc tgagccacgt gagcatgttc cgcagtggct tcagcaacag
cagcgtgagc atcatccgtg cacctatgtt cagctggatt caccgcagtg ccgagttcaa
caacatcatc cccagcagcc agatcaccca gatccccctg accaagagca ccaacctggg
cagcggcacc agcgtggtga agggccccgg cttcaccggc ggcgacatcc tgcgccgcac
cagccccggc cagatcagca ccctgcgcgt gaacatcacc gcccccctga gccagcgcta
ccgcgtccgc atccgctacg ccagcaccac caacctgcag ttccacacca gcatcgacgg
ccgccccatc aaccagggca acttcagcgc caccatgagc agcggcagca acctgcagag
cggcagcttc cgcaccgtgg gcttcaccac ccccttcaac ttcagcaacg gcagcagcgt
gttcaccctg agcgcccacg tgttcaacag cggcaacgag gtgtacatcg accgcatcga
gttcgtgccc gccgaggtga ccttcgaggc cgagtacgac ctggagaggg ctcagaaggc
cgtgaacgag ctgttcacca gcagcaacca gatcggcctg aagaccgacg tgaccgacta
ccacatcgat caggtgcgag gccccggtgg agcaggtgga gcaggtggag ggggtaaagg
aaacaacacc aagctgaacg ccagggccct gccgagcttc atcgactact tcaacggcat
ctacggcttc gccaccggca tcaaggacat catgaacatg atcttcaaga ccgacaccgg
cggcaacctg accctggacg agatcctgaa gaaccagcag ctgctgaacg agatcagcgg
caagctggac ggcgtgaacg gcagcctgaa cgacctgatc gcccagggca acctgaacac
cgagctgagc aaggagatcc tgaagatcgc caacgagcag aaccaggtgc tgaacgacgt
gaacaacaag ctggacgcca tcaacaccat gctgcacatc tacctgccga agatcaccag
catgctgagc gacgtgatga agcagaacta cgccctgagc ctgcaggtgg agtacctgag
caagcagctg aaggagatca gcgacaagct ggacgtgatc aacgtgaacg tgctgatcaa
cagcaccctg accgagatca ccccggccta ccagaggatc aagtacgtga acgagaagtt
cgaggagctg accttcgcca ccgagaccac cctgaaggtg aagaaggaca gcagcccggc
cgacatcctg gacgagctga ccgagctgac cgagctggcc aagagcgtga ccaagaacga
cgtggacggc ttcgagttct acctgaacac cttccacgac gtgatggtgg gcaacaacct
gttcggcagg agcgccctga agaccgccag cgagctgatc gccaaggaga acgtgaagac
cagcggcagc gaggtgggca acgtgtacaa cttcctgatc gtgctgaccg ccctgcaggc
caaggccttc ctgaccctga ccacctgcag gaagctgctg ggcctggccg acatcgacta
caccagcatc atgaacgagc acctgaacaa ggagaaggag gagttcaggg tgaacatcct
gccgaccctg agcaacacct tcagcaaccc gaactacgcc aaggtgaagg gcagcgacga
ggacgccaag atgatcgtgg aggccaagcc gggccacgcc ctggtgggct tcgagatcag
caacgacagc atgaccgtgc tgaaggtgta cgaggccaag ctgaagcaga actaccaggt
ggacaaggac agcctgagcg aggtgatcta cagcgacatg gacaagctgc tgtgcccgga
ccagagcgag cagatctact acaccaacaa catcgtgttc ccgaacgagt acgtgatcac
caagatcgac ttcaccaaga agatgaagac cctgaggtac gaggtgaccg ccaacagcta
cgacagcagc accggcgaga tcgacctgaa caagaagaag gtggagagca gcgaggccga
gtacaggacc ctgagcgcca acaacgacgg cgtgtacatg ccgctgggcg tgatcagcga
gaccttcctg accccgatca acggcttcgg cctgcaggcc gacgagaaca gcaggctgat
caccctgacc tgcaagagct acctgaggga gctgctgctg gccaccgacc tgagcaacaa
ggagaccaag ctgatcgtgc cgccgatcag cttcatcagc aacatcgtgg agaacggcaa
cctggagggc gagaacctgg agccgtggat cgccaacaac aagaacgcct acgtggacca
caccggcggc atcaacggca ccaaggtgct gtacgtgcac aaggacggcg agttcagcca
gttcgtgggc ggcaagctga agagcaagac cgagtacgtg atccagtaca tcgtgaaggg
caaggccagc atctacctga aggacgagaa caccggctac atccactacg aggacaccaa
caacaacctg gaggactacc agaccatcaa caagaggttc accaccggca ccgacctgaa
gggcgtgtac ctgatcctga agagccagaa cggcgacgag gcctggggcg acaacttcat
catcctggag atcagcccga gcgagaagct gctgagcccg gagctgatca acaccaacaa
ctggaccagc accggcagca ccaacatcag cggcaacacc ctgaccctgt accagggcgg
caggggcatc ctgaagcaga acctgcagct ggacagcttc agcacctaca gggtgtactt
cagcgtgagc ggcgacgcca acgtgaggat caggaacagc agggaggtgc tgttcgagaa
gaggtacatg agcggcgcca aggacgtgag cgagatgttc accaccaagt tcgagaagga
caacttctac atcgagctga gccagggcaa caacctgtac ggcggcccga tcgtgcactt
ctacgacgtg agcatcaagt aagagctctt caaacatttg gcaataaagt ttcttaagat
tgaatcctgt tgccggtctt gcgatgatta tcatataatt tctgttgaat tacgttaagc
atgtaataat taacatgtaa tgcatgacgt tatttatgag atgggttttt atgattagag
tcccgcaatt atacatttaa tacgcgatag aaaacaaaat atagcgcgca aactaggata
aattatcgcg cgcggtgtca tctatgttac tagatcggta ccgagctcga attcgtaatc
atggtcatag ctgtttcctg tgtgaaattg ttatccgctc acaattccac acaacatacg
agccggaagc ataaagtgta aagcctgggg tgcctaatga gtgagctaac tcacattaat
tgcgttgcgc tcactgcccg ctttccagtc gggaaacctg tcgtgccagc tgcattaatg
aatcggccaa cgcgcgggga gaggcggttt gcgtattggc tagagcagct tgccaacatg
gtggagcacg acactctcgt ctactccaag aatatcaaag atacagtctc agaagaccaa
agggctattg agacttttca acaaagggta atatcgggaa acctcctcgg attccattgc
ccagctatct gtcacttcat caaaaggaca gtagaaaagg aaggtggcac ctacaaatgc
catcattgcg ataaaggaaa ggctatcgtt caagatgcct ctgccgacag tggtcccaaa
gatggacccc cacccacgag gagcatcgtg gaaaaagaag acgttccaac cacgtcttca
aagcaagtgg attgatgtga taacatggtg gagcacgaca ctctcgtcta ctccaagaat
atcaaagata cagtctcaga agaccaaagg gctattgaga cttttcaaca aagggtaata
tcgggaaacc tcctcggatt ccattgccca gctatctgtc acttcatcaa aaggacagta
gaaaaggaag gtggcaccta caaatgccat cattgcgata aaggaaaggc tatcgttcaa
gatgcctctg ccgacagtgg tcccaaagat ggacccccac ccacgaggag catcgtggaa
aaagaagacg ttccaaccac gtcttcaaag caagtggatt gatgtgatat ctccactgac
gtaagggatg acgcacaatc ccactatcct tcgcaagacc ttcctctata taaggaagtt
catttcattt ggagaggaca cgctgaaatc accagtctct ctctacaaat ctatctctct
cgagtcaaca caacatatac aaaacaaacg aatctcaagc aatcaagcat tctacttcta
ttgcagcaat ttaaatcatt tcttttaaag caaaagcaat tttctgaaaa ttttcaccat
ttacgaacga tagccatggc tcaagttagc agaatctgca atggtgtgca gaacccatct
cttatctcca atctctctaa atccagtcaa aggaaatctc ccttatcggt ttctctgaag
actcagcagc atccacgagc ttatccaatt tcttcatctt ggggattgaa gaagagtggg
atgactttaa ttggctctga gcttcgtcct cttaaggtca tgtcttctgt ttccacggcg
gagaagggat ccgacgctct tccagctacc ttcgacgtta tcgtgcatcc agctagagaa
ctcagaggtg aacttagagc acagccatcc aagaactaca ccactagata cctcctcgcc
gctgctctcg ctgagggtga aaccagagtt gttggtgtgg ctacctctga ggatgccgaa
gctatgctca gatgcctcag agattggggt gctggtgttg agcttgttgg tgatgacgcc
gtgatcagag gtttcggtgc tagaccacag gctggtgtta cccttaaccc aggtaacgct
gctgcggtgg ccagactcct tatgggtgtt gctgctctca cctctggtac aactttcgtt
accgattacc ctgattccct tggtaagaga cctcagggtg accttcttga agccctcgaa
agacttggtg cttgggtgtc ctccaacgat ggtagactcc ctatctccgt ttccggtcca
gttagaggtg gtacagtgga ggtttccgcc gaaagatcct cccagtacgc ttccgccctt
atgttcctcg gtcctcttct tcctgacgga ctcgaactta gactcaccgg tgatatcaag
tcccacgctc ctcttagaca gacacttgac accctctctg atttcggtgt tagagctact
gcctccgatg accttagaag aatctccatc cctggtggtc agaagtacag accaggtaga
gtgctcgttc ctggtgatta ccctggttcc gctgctatcc ttaccgccgc tgctcttctc
ccaggtgagg ttagactttc taaccttaga gaacacgacc tccagggtga gaaggaagct
gtgaacgttc ttagagagat gggtgctgat atcgttagag aaggtgatac ccttaccgtg
agaggtggta gacctctcca cgctgttact agagatggtg attccttcac cgacgccgtg
caagctctta ccgctgctgc tgccttcgct gagggtgata ccacctggga aaacgttgct
actcttagac tcaaggaatg cgatagaatc tctgacacca gagctgagct tgaaagactt
ggtcttagag caagagagac cgccgattct ctctccgtta ctggttctgc tcaccttgct
ggtggtatca ccgctgatgg tcacggtgac cacagaatga tcatgcttct cacccttctt
ggtctcagag cagatgctcc acttagaatc accggtgcac accacatcag aaagtcctac
cctcagttct tcgctcacct tgaagctctt ggtgctagat tcgaatacgc tgaggctacc
gcctaatagg agctcgagtt tctccataat aatgtgtgag tagttcccag ataagggaat
tagggttcct atagggtttc gctcatgtgt tgagcatata agaaaccctt agtatgtatt
tgtatttgta aaatacttct atcaataaaa tttctaattc ctaaaaccaa aatccagtac
taaaatccag atcccccgaa ttaattcggc gttaatccag tacattaaaa acgtccgcaa
tgtgttatta agttgtccac tattattgtt tttgtattat attttcccag gcttatcttt
tatcaatgag ttatatataa agaaataatc agtcaacatg tagcaacaaa atatttgcag
taataataat aacgttaaac aatagaaatt aaaaaaacct aacaacaaat gtcttgattt
taaagacttg tgttcacatg atcatttgat caagtaaaag ataacttcct aaggcaagaa
tgaaagtagt gagatgtttt tagttttttt tttccacaca aactaattct aaaaacattt
tatcttaaac aaaaatacaa ttaacaaaga aaatattttt caacaatctc atactaatgt
aaaacttcag gaaatacata atataataaa aacattttta atggaacatc atgcattgtg
ttttcatcca ttaatttttc atgcttacta gaaaggaact taatcatatc cattatagat
attttgatac aacatcatgc taccgcagaa aagactatgc aagaagaaag tataaaacat
ttttctctct taagactgtt ggaaaataaa acaaaaatga aggaaaataa atacgaagaa
gatgcacagt cttgaatta.

The SEQ ID NO:10 or its complementary sequence is a 9559-nucleotide sequence that represents the transgenic soybean event CAL16. The specific genomic and genetic elements contained in SEQ ID NO:10 are listed in Table 1. The presence of SEQ ID NO:10 or its complementary sequence can be used to identify the existence of the transgenic soybean event CAL16. The present invention provides a continuous nucleotide sequence specific to the transgenic soybean event CAL16. This continuous nucleotide sequence can be used to characterize the transgenic soybean event CAL16 and detect its presence in samples. Specifically, the presence of at least 11 consecutive nucleotides from any one or more of the nucleic acid molecules shown in SEQ ID NO:1-10 in the sample indicates the presence of the transgenic soybean event CAL16.

In the present invention, the first nucleotide sequence used to detect the transgenic soybean event CAL16 in a sample can be SEQ ID NO:7 or its complementary sequence and/or SEQ ID NO:8 or its complementary sequence and/or SEQ ID NO:9 or its complementary sequence, comprising at least 11 or more consecutive nucleotides from any portion of the transgene insertion sequence. The second nucleotide sequence can be at least 11 or more consecutive nucleotides from any portion of the 5′ flanking genomic DNA region of the soybean genome, which can be SEQ ID NO:7 or its complementary sequence. When the first nucleotide sequence and the second nucleotide sequence are used together, these nucleotide sequences form DNA primer pairs in DNA amplification methods that generate amplified products. The amplified product comprising SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10 can be used to diagnose the presence of the transgenic soybean event CAL16 or its progeny. It is known to those skilled in the art that the first and second nucleotide sequences can be composed not only of DNA but also RNA, mixtures of DNA and RNA, or combinations of DNA, RNA, or other nucleotides or analogs thereof that are not templates for polymerases. Additionally, the probes or primers used for detection in the present invention can be selected from the nucleotides described in SEQ ID NO:1, SEQ ID NO:2, SEQ ID NO:3, SEQ ID NO:4, SEQ ID NO:5, or SEQ ID NO:6, and can have a length of at least 11, 12, 13, 14, 15, 16, 17, 18, 19, 20, 21, or 22 consecutive nucleotides. When selected from the nucleotides shown in SEQ ID NO:7, SEQ ID NO:8, SEQ ID NO:9, or SEQ ID NO:10, the probes and primers can have a length of at least approximately 21 to approximately 50 or more consecutive nucleotides.

The nucleotide sequence or its complementary sequence can be used in DNA amplification methods to generate amplicons, which are used for detecting and diagnosing the presence of the transgenic soybean event CAL16 or its progeny in biological samples. The nucleotide sequence or its complementary sequence can also be used in nucleic acid detection methods to detect the presence of the transgenic soybean event CAL16 or its progeny in biological samples.

The third aspect of the present invention provides a method for detecting the presence of the transgenic soybean event CAL16 DNA molecules in a sample. The method includes the following steps: (1) contacting the sample to be tested with DNA probes or primer pairs in a nucleic acid amplification reaction mixture. The primer pair comprises a first primer and a second primer. The first primer can be either SEQ ID NO:23 or SEQ ID NO:25, and the second primer can be either SEQ ID NO:22 or SEQ ID NO:26. The DNA probe is shown as SEQ ID NO:24. (2) Performing a nucleic acid amplification reaction. (3) Detecting the presence of amplified products, which include at least 11 consecutive nucleotides from SEQ ID NO:1 or its complementary sequence and/or SEQ ID NO:2 or its complementary sequence. The probe is labeled with at least one fluorescent group, preferably 6FAMTM (6-carboxyfluorescein).

Preferably, the amplified product includes at least 11 consecutive nucleotides from SEQ ID NO:3 or its complementary sequence, and/or SEQ ID NO:4 or its complementary sequence.

Furthermore, preferably the amplified product includes at least 11 consecutive nucleotides from SEQ ID NO:5 or its complementary sequence, and/or SEQ ID NO:6 or its complementary sequence.

Additionally, preferably the amplified product includes at least 11 consecutive nucleotides from SEQ ID NO:7 or its complementary sequence, and/or SEQ ID NO:8 or its complementary sequence.

Furthermore, the amplified product includes at least 11 consecutive nucleotides from SEQ ID NO:1 or its complementary sequence. SEQ ID NO:2 or its complementary sequence, SEQ ID NO:3 or its complementary sequence, SEQ ID NO:4 or its complementary sequence, SEQ ID NO:5 or its complementary sequence, SEQ ID NO:6 or its complementary sequence, SEQ ID NO:7 or its complementary sequence, and/or SEQ ID NO:8 or its complementary sequence, and/or SEQ ID NO:9 or its complementary sequence, and/or SEQ ID NO:10 or its complementary sequence.

The primer pairs in the present invention include at least one DNA primer sequence derived from SEQ ID NO:7, SEQ ID NO:8, SEQ ID NO:9, or SEQ ID NO:10. Primer SEQ ID NO:22 has the same nucleotide sequence as positions 8929 to 8954 of SEQ ID NO:10, positions 999 to 1024 of SEQ ID NO:8, and positions 9 to 34 of SEQ ID NO:6. Primer SEQ ID NO:23 has the reverse complementary nucleotide sequence to positions 9042 to 9069 of SEQ ID NO:10 and positions 1112 to 1139 of SEQ ID NO:8. Probe sequence (SEQ ID NO:24) is identical to the nucleotide sequence at positions 8996 to 9010 of SEQ ID NO:10, positions 1066 to 1080 of SEQ ID NO:8, and positions 76 to 95 of SEQ ID NO:6Primer SEQ ID NO:25 has the same nucleotide sequence as positions 296 to 323 of SEQ ID NO:10 and positions 296 to 323 of SEQ ID NO:7. Primer SEQ ID NO:26 has the reverse complementary nucleotide sequence to positions 500 to 525 of SEQ ID NO:10, positions 57 to 82 of SEQ ID NO:9, and positions 500 to 525 of SEQ ID NO:7.

The fourth aspect of the present invention provides a method for cultivating insect-resistant soy bean plants containing the transgenic soybean event CAL16. The method comprises the following steps: planting soybean seeds containing specific nucleotide sequences, harvesting soybeans with significantly improved resistance to Lepidopteran insects compared to soybean plants without the specific nucleotide sequences, and protecting the soybean plants from insect infestation. The specific nucleotide sequences are selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or their complementary sequences. The Lepidopteran insects include but are not limited to Fall armyworm, Beet armyworm, Cotton bollworm, and Soybean looper.

The fifth aspect of the present invention provides a method for cultivating herbicide-tolerant soy bean plants containing the transgenic soy bean event CAL16. The method comprises the following steps: planting soybean seeds containing specific nucleotide sequences, applying herbicides, and harvesting soybeans with significantly improved tolerance to herbicides compared to soybean plants without the specific nucleotide sequences. The specific nucleotide sequences are selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or their complementary sequences. The herbicide used includes glyphosate.

The sixth aspect of the present invention provides a method for controlling field weeds in soybean plants containing the transgenic soybean event CAL16. The method comprises the following steps: planting transgenic soybean plants containing specific region nucleotide sequences, applying an effective dose of the herbicide glyphosate, and killing the weeds. The specific region nucleotide sequences are derived from the transgenic event CAL16 and include SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or their complementary sequences present in the transgenic soybean genome.

The seventh aspect of the present invention provides a method for generating soybean plants with insect resistance and/or glyphosate tolerance. The method comprises the following steps: crossing soy bean plants containing specific region nucleotide sequences with another soybean plant to generate progeny plants, and harvesting plants that show significantly improved tolerance to herbicides and/or resistance to insects compared to plants lacking the specific region nucleotide sequences. The specific region nucleotide sequences are derived from the transgenic soybean event CAL16 and include SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or their complementary sequences.

The eighth aspect of the present invention provides transgenic plant cells generated from the transgenic soybean event CAL16. The described transgenic plant cells are obtained by introducing the transgenic soybean event CAL16 into the plant genome. The specific region nucleotide sequences include SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or their complementary sequences into the plant genome.

The ninth aspect of the present invention provides commercial or agricultural products derived from the transgenic soybean event CAL16 soybeans. The soybean products may include soy bean oil, soy protein, soy bean meal, soy flour, soybean flakes, soybean skins, soy milk, soy cheese, soybean wine, animal feed containing soy beans, paper containing soy beans, soy-based cheese, soy biomass, and fuel products produced from soybean plants and soybean plant parts.

The term “soybean” refers to Glycine max, including all plant varieties that can be bred using soy bean plants containing the transgenic soybean event CAL16. This includes wild soybean species as well as plants within the genus Glycine that are amenable to breeding. The term “including” means “including but not limited to.”

The term “flanking DNA” can include both naturally occurring genomic DNA present in organisms such as plants, as well as exogenous (foreign) DNA introduced through transformation processes, such as fragments associated with the transgenic event. Therefore, flanking DNA can encompass a combination of natural and exogenous DNA. In the present invention, the terms “flanking region,” “flanking sequence,” “genome boundary region,” or “genome boundary sequence” refer to a sequence of at least 3, 5, 10, 11, 15, 20, 50, 100, 200, 300, 400, 1000, 1500, 2000, 2500, or 5000 base pairs or longer, which is located directly upstream or downstream of the original exogenous inserted DNA molecule and is adjacent to the original exogenous inserted DNA molecule. When the flanking region is located downstream, it can also be referred to as the “left flanking region.” “3′ flanking region,” “3′ genome boundary region,” or “genome 3′ boundary sequence,” etc. When the flanking region is located upstream, it can also be referred to as the “right flanking region.” “5′ flanking region,” “5′ genome boundary region.” or “genome 5′ boundary sequence,” etc.

The random integration of exogenous DNA during transformation procedures can lead to transformation events with different flanking regions, which are specific to each transformation event. When recombinant DNA is introduced into plants through traditional hybridization methods, the flanking regions usually remain unchanged. Transformation events also contain unique junctions between segments of exogenous insert DNA and genomic DNA, or between two segments of genomic DNA, or between two segments of exogenous DNA. The “junction” refers to the point of connection between two specific DNA fragments. For example, the junction exists at the location where the insert DNA is connected to the flanking DNA. Junction points also exist in the transformed organism, where two DNA fragments are connected together in a manner that is modified from what is naturally found in the organism. “Junction DNA” refers to DNA that contains the junction point.

The present invention, transgenic soybean event CAL16, exhibits superior characteristics and performance compared to existing transgenic soybean plants and concurrently constructed new events. It includes a DNA construct that is inserted into the soybean genome in a single form.

The DNA construct (FIG. 1) comprises a T-DNA segment containing two linked plant expression cassettes, wherein the regulatory elements drive the expression of insecticidal protein Cry1Ab/Vip3Da and the glyphosate-tolerant G10evo EPSPS in soybean plant cells. The DNA segment encodes a fusion protein of two different insecticidal proteins, Cry1Ab-Vip3Da, expressed by the inserted transgenic DNA in the expression cassette as listed in SEQ ID NO:10 and shown in FIG. 1. The Cry1Ab-Vip3Da gene cassette is composed of the pCsVMV promoter, the insecticidal fusion gene cry1Ab-Vip3Da, and the NOS terminator derived from Agrobacterium. The pCsVMV promoter is a constitutive promoter derived from the figwort mosaic virus, which can drive the expression of the target gene in all plant tissues, and the terminator is the NOS terminator derived from Agrobacterium. The DNA segment encodes the 5-enolpyruvylshikimate-3-phosphate synthase G10evo EPSPS, which confers glyphosate tolerance, expressed by the inserted transgenic DNA in the expression cassette as listed in SEQ ID NO:10 and shown in FIG. 1. The g10evo epsps gene cassette is composed of the 35S promoter, TEV 5′UTR, Arabidopsis EPSPS signal peptide, g10evo epsps gene, and 35S terminator. The 35S promoter is a constitutive promoter derived from the cauliflower mosaic virus, which can drive the expression of the target gene in all plant tissues. The chloroplast signal peptide is derived from Arabidopsis, and the g10evo epsps gene is derived from the radiation-resistant bacterium Deinococcus radiodurans. The terminator is the 35S terminator derived from the cauliflower mosaic virus.

The DNA construct is introduced into the soybean genome using Agrobacterium-mediated transformation of soy bean cotyledonary nodes.

The present invention provides exemplary primers or probes that can be used to detect the presence of DNA derived from soybean plants containing the CAL16 event in a sample. These primers or probes are specific to the target nucleic acid sequence and are therefore suitable for identifying the CAL16 nucleic acid sequence using the methods described in this disclosure.

The term “probe” refers to a single-stranded nucleic acid that is complementary to the target nucleic acid. In the context of the present invention, the probe not only includes deoxyribonucleic acid (DNA) or ribonucleic acid (RNA), but also may contain polymers and other probe materials that specifically bind to the target DNA sequence. Detection of such binding can be used for diagnosis, differentiation, quantification, or confirmation of the presence of the target DNA sequence in a specific sample. The probe can be labeled or tagged with conventional detectable markers or reporting molecules, such as radioactive isotopes, ligands, chemiluminescent agents, or enzymes. An exemplary DNA molecule that can be used as a probe is provided as SEQ ID NO:24.

The term “primer” refers to a highly purified, isolated nucleotide sequence that is designed for use in specific annealing or hybridization methods involving thermal amplification. A pair of primers can be used together with template DNA (such as a sample of soybean genomic DNA) in thermal amplification techniques such as polymerase chain reaction (PCR) to generate amplicons. The amplicons produced from such reactions will have DNA sequences corresponding to the template DNA sequence between the two sites where the primers hybridize to the template. In this context, an “amplicon” refers to a replicated fragment of DNA synthesized using amplification techniques. The amplicons of the present invention can include at least one sequence provided by SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:10. Primers are typically designed to hybridize to the complementary target DNA strand, forming hybrids between the primer and the target DNA strand, and the presence of primers is recognized by the polymerase as the starting point for primer extension using the target DNA strand as a template (i.e., additional nucleotides are polymerized into the growing nucleotide molecule). The primer pairs used in the present invention are designed to indicate the use of double-stranded nucleotide segments, where the two primers bind to opposite strands, so that a segment of multiple nucleotides between the positions targeted by the primer pairs can be linearly amplified in thermal amplification reactions or other conventional nucleic acid amplification methods. Exemplary DNA molecules that can be used as primers are provided as SEQ ID NO:22, SEQ ID NO:23, SEQ ID NO:25, or SEQ ID NO:26. The primer pairs provided by SEQ ID NO:25 and SEQ ID NO:26 are intended for use with a first DNA molecule and a second DNA molecule that is different from the first DNA molecule, both having a contiguous nucleotide sequence of SEQ ID NO:10 of sufficient length to serve as DNA primers. These DNA primers, when used together with template DNA derived from the soybean event CAL16 in a thermal amplification reaction, generate amplicons of the CAL16 DNA for diagnostic purposes in the sample.

According to the present invention, probes and primers have complete sequence identity with the target sequence while still retaining the ability to preferentially hybridize to the target sequence. Probes and primers that are different from the target sequence can be designed using conventional methods. To be used as probes or primers, nucleic acid molecules only need to be sufficiently complementary in sequence to form a stable duplex structure under specific solvent and salt concentration conditions. Any conventional nucleic acid hybridization or amplification methods can be used to identify the presence of transgenic DNA from the soybean event CAL16 in a sample. Probes and primers are generally at least about 11, 18, 24, or 30 nucleotides long or longer. Such probes and primers specifically hybridize to the target DNA sequence under stringent hybridization conditions. The conventional stringent conditions are described by Sambrook et al., 1989, and by Haymes et al, in “Nucleic Acid Hybridization, A Practical Approach,” IRL Press, Washington, DC (1985).

In the present invention, the term ‘amplified DNA’ or ‘amplicon’ refers to the nucleic acid amplification product of the target nucleic acid sequence, which is part of the nucleic acid template. For example, in order to determine whether soybean plants are derived from the transgenic soybean event CAL16 of the present invention, produced by sexual hybridization or to verify if soybean samples collected from the field contain the transgenic soybean event CAL16, or whether soybean extracts such as crude powder, flour, or oil contain the transgenic soybean event CAL16, DNA extracted from soybean plant tissue samples or extracts can be used as a diagnostic amplicon for the presence of DNA specific to the transgenic soybean event CAL16 by using primer pairs in nucleic acid amplification methods. The primer pair comprises a first primer derived from a flanking sequence adjacent to the insertion site of the exogenous DNA in the plant genome, and a second primer derived from the exogenous DNA insertion. The amplicon has a certain length and sequence that are diagnostic for the transgenic soybean event CAL16. The length range of the amplicon can be the binding length of the primer pair plus one base pair, preferably plus approximately fifty base pairs, more preferably plus approximately two hundred and fifty base pairs, most preferably plus approximately four hundred and fifty base pairs or more.

Many methods known to those skilled in the art can be used to isolate and manipulate the DNA molecules or fragments disclosed in the present invention, including the polymerase chain reaction (PCR) method. DNA molecules or fragments can also be obtained through other techniques, such as chemical synthesis of fragments directly, for example, using an automated oligonucleotide synthesizer.

The term ‘offspring or progeny’ as used herein encompasses any plants, seeds, plant cells, and/or regenerable plant parts that contain DNA molecules derived from the ancestral plant of event CAL16 and/or contain at least one sequence selected from the following group of compositions: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10. Plants, offspring, and seeds can be homozygous or heterozygous for the transgene. Offspring can grow from seeds produced by plants containing the soybean event CAL16 and/or from seeds produced by plants pollinated with pollen from plants containing the soybean event CAL16. Progeny plants can be generated through self-pollination (also known as ‘selfing’) to create true plant breeding lines that are homozygous for the transgene. Appropriate selfing of the progeny can produce plants that are homozygous for the introduced exogenous gene. Alternatively. progeny plants can undergo outcrossing, such as breeding with another unrelated plant, to generate variety or hybrid seeds or plants. The unrelated plant can be either transgenic or non-transgenic. Thus, varieties or hybrid seeds or plants of the present invention can be obtained by crossing a first parent lacking the specific and unique DNA of soybean event CAL16 with a second parent containing soybean event CAL16 in a sexual manner, resulting in hybrids that contain the specific and unique DNA of soy bean event CAL16. Each parent can be a hybrid or inbred line/variety, as long as the crossbreeding or breeding results in plants or seeds of the present invention, which have at least one allele of DNA containing soybean event CAL16 and/or at least one sequence selected from the following: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10. Two different transgenic plants can crossbreed to produce hybrid offspring containing two independent, introgressed, exogenous genes. For example, a plant containing CAL16 with a dual mode of insect resistance and glyphosate tolerance can hybridize with another transgenic soybean plant possessing additional traits such as herbicide tolerance and/or insect control, resulting in offspring plants or seeds with dual resistance against Lepidopteran insect pests and at least one or more additional traits. Backcrossing to parent plants and outcrossing to non-transgenic plants, as well as asexual reproduction, can also be performed. Descriptions of other breeding methods for different traits and crops can be found in various references, such as Fehr, Breeding Methods for Cultivar Development, edited by J. Wilcox, American Society of Agronomy, Madison, WI (1987).

The term ‘transgenic plant cell’ is applicable to many industrial applications, including but not limited to: (i) serving as research tools for scientific exploration or industrial research; (ii) being used in cultures for the production of endogenous or recombinant carbohydrates, lipids, nucleic acids, proteins, or small molecules, which can be further used for scientific research or as industrial products; and (iii) being used in conjunction with modern plant tissue culture techniques for the production of transgenic plants or plant tissue cultures, which can subsequently be used for agricultural research or production. Microbial production and utilization of transgenic plant cells involve modern microbiological techniques and artificial interventions to generate engineered and unique microbes. In this process, recombinant DNA is inserted into the plant cell genome to generate transgenic plant cells that are distinct and unique from naturally occurring plant cells. These transgenic plant cells can then be cultured using modern microbiological techniques similar to bacteria and yeast cells and can exist in an undifferentiated single-cell state. The novel genetic composition and phenotype of transgenic plant cells are the result of the technology of integrating exogenous DNA into the cell's genome. Another aspect of the present invention is a method utilizing the microorganisms of the present invention. Methods of using the microorganisms of the present invention, such as transgenic plant cells, include (i) generating transgenic cells by integrating recombinant DNA into the genome of the cells, followed by using these cells to obtain additional cells with the same exogenous DNA; (ii) culturing cells containing recombinant DNA using modern microbiological techniques; (iii) producing and purifying endogenous or recombinant carbohydrates, lipids, nucleic acids, or protein products from cultured cells; and (iv) using transgenic plant cells in conjunction with modern plant tissue culture techniques to produce transgenic plants or transgenic plant tissue cultures.

The term ‘commercial product’ refers to any combination or product composed of materials originating from soybean plants containing the CAL16 DNA, including but not limited to soybean plants, whole or processed soybean seeds, one or more plant cells, and/or plant parts. Commercial products can be sold to consumers and can be either live or non-living. Non-living commercial products include, but are not limited to, non-viable seeds; whole or processed seeds, seed parts, and plant parts; soybean oil, soy bean protein, soybean meal, soybean flour, soybean grits, soybean hulls, soy milk, soy cheese, soy sauce, animal feed containing soybean, paper containing soybean, cheese containing soybean, soy biomass, and fuel products produced using soybean plants and soybean plant parts. Live commercial products include, but are not limited to, seeds, plants, and plant cells. Therefore, soybean plants containing the CAL16 event can be used to manufacture any commercial product typically obtained from soybeans. Any such commercial product derived from soybean plants containing the CAL16 event may contain a detectable amount of specific and unique DNA corresponding to the CAL16 event, and specifically, may contain a detectable amount of nucleic acids that include at least one sequence selected from the following: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

Compared to the prior art, the present invention has the following beneficial effects:
(1) The present invention provides a transgenic soybean event CAL16. The CAL16 event in the transgenic soybean provides resistance against Lepidopteran pests and tolerance to glyphosate-based herbicides. The genes encoding insect resistance and glyphosate tolerance traits are linked in the same DNA region and present at a single locus in the genome of the transgenic soybean event CAL16. This provides enhanced breeding efficiency and allows for the use of molecular markers to track the transgenic insertion fragments in breeding populations and their progeny. The transgenic soybean event CAL16 is preserved in the China Center for Type Culture Collection (CCTCC) with the accession number CCTCC NO: P202205, deposited on Apr. 18, 2022, at Wuhan University, Wuhan, China, postal code 430072.
(2) The present invention provides specific nucleotide sequences for detecting soybean plants and their detection methods. The specific detection method provided in the present invention, using SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10 or their complementary sequences, allows for the specific detection of the transgenic soybean event CAL16.
(3) The present invention provides specific nucleotide sequences for detecting soybean plants and their detection methods. Specifically designed primers or probes targeting the specific sequences SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10 or their complementary sequences can be used as DNA primers or probes to generate amplification products that diagnose the presence of the transgenic soybean event CAL16 or its progeny. This allows for rapid, accurate, and stable identification of plant materials originating from the transgenic soybean event CAL16. It enables traceability and full-process supervision of research, production, processing, and applications related to CAL16.
(4) The present invention provides methods for obtaining progeny, agricultural products, or commercial products containing the transgenic soybean event CAL16.

### BREIF DESCRIPTION OF THE DRAWINGS

FIG. 1: Schematic diagram of the transformation construct used to generate the transgenic soy bean event.

FIG. 2: Results of bioassays on T0 generation transformation events for cotton bollworm, a. Transgenic event CAL16; b. Transgenic event CAL29; c. Transgenic event CAL13; d. Transgenic event CAL39; e. Transgenic event CAL56; f. Non-transgenic soybean variety Tianlong-1.

FIG. 3: PCR analysis for the identification of any tissues with breeding activity containing the transgenic soybean event CAL16. M: Marker; 1: Seeds of transgenic soybean event CAL16; 2: Leaves of transgenic soybean event CAL16; 3: Pods of transgenic soybean event CAL16; 4: Negative control; 5: Non-transgenic soybean variety Tianlong-1; 6: Transgenic cotton with insect resistance; 7: Conventional rice.

### SPECIFIC EMBODIMENT

The present invention will be further described in conjunction with specific embodiments. It should be understood by those skilled in the art that the examples provided herein represent preferred embodiments of the invention, as discovered by the inventors in practicing the present invention, and can be considered to constitute preferred embodiments for implementing the invention. However, it should be understood that numerous modifications can be made to the specific embodiments disclosed herein, based on the teachings provided in this disclosure, to achieve similar or equivalent results without departing from the spirit and scope of the invention. The scope of the invention is not limited to the embodiments described herein.

The molecular biology and biochemical methods used in the following embodiments are well-known techniques. Detailed explanations of these techniques can be found in references such as “Current Protocols in Molecular Biology” published by John Wiley and Sons, Inc., edited by Ausubel, and “Molecular Cloning: A Laboratory Manual, 3rd Edition” published by Cold Spring Harbor Laboratory Press (2001), edited by J. Sambrook et al.

The specific formulations of the media used in the following embodiments of the present invention are as follows:
YEP solid medium composition: Trytone (peptone) 10 g/L, Yeast extract 10 g/L, Sodium chloride 5 g/L, Agar 2.8 g/L, solvent is water, pH 7.0.
Germination medium composition: MS salt (Phytotech M524) 4.33 g/L, Sucrose 20 g/L, Agar 2.75 g/L, solvent is water, pH 5.8.
GADT liquid medium composition: B5 salt (Phytotech G398) 0.32 g/L, 2-(N-morpholino) ethanesulfonic acid (MES) 3.9 g/L, Sucrose 30 g/L, solvent is water, pH 5.4. After sterilization by high-temperature and high-pressure, and cooling, the following filter-sterilized compounds are added to the medium: 0.835 mg/L of 6-benzylaminopurine (6-BA), 0.25 mg/L of gibberellic acid A3 (GA3), 40 mg/L of acetosyringone (AS), 154 mg/L of DL-dithiothreitol (DTT), 1 mM of sodium metabisulfite(S), and 2.4 g/L of cysteine (Cys).
Recovery medium composition: B5 salt (Phytotech G398) 3.21 g/L, MES 0.6 g/L, Sucrose 30 g/L, Agar 2.8 g/L, solvent is water, pH 5.7. After sterilization by high-temperature and high-pressure, and cooling, the following filter-sterilized compounds are added to the medium: 0.835 mg/L of 6-benzylaminopurine (6-BA) and 200 mg/L of Timentin.
Screening medium composition: B5 salt (Phytotech G398) 3.21 g/L, MES 0.6 g/L, Sucrose 30 g/L, Agar 2.8 g/L, solvent is water, pH 5.7. After sterilization by high-temperature and high-pressure, and cooling, the following filter-sterilized compounds are added to the medium: 0.835 mg/L of 6-benzylaminopurine (6-BA), 200 mg/L of Timentin, and 25 mg/L of glyphosate.
Elongation medium composition: MS salt and B5 vitamin mixture (Phytotech M404) 4.44 g/L, MES 0.59 g/L, Glutamine 0.05 g/L, Asparagine 0.05 g/L, Sucrose 30 g/L, Agar 2.8 g/L, solvent is water, pH 5.7. After sterilization by high-temperature and high-pressure, and cooling, the following filter-sterilized compounds are added to the medium: 200 mg/L of Timentin, 25 mg/L of glyphosate, 0.25 mg/L of gibberellic acid A3 (GA3), 1 mg/L of zeatin, and 0.1 mg/L of indole-3-acetic acid (IAA).
Rooting medium composition: MS salt and B5 vitamin mixture (Phytotech M404) 4.44 g/L, MES 0.59 g/L, Sucrose 30 g/L, Agar 2.8 g/L, solvent is water, pH 5.7. After sterilization by high-temperature and high-pressure, and cooling, the following filter-sterilized compounds are added to the medium: 200 mg/L of Timentin, 25 mg/L of glyphosate, and 0.1 mg/L of IAA.

### Example 1: Acquisition of Transgenic Events

### (1) Acquisition of Plasmid Vector Containing Exogenous Genes

The plasmid vector pCAL, used for soybean transformation in the present invention, is depicted in FIG. 1. The pCAL vector is based on the plant transformation vector framework pCambial300 (GenBank: AF234296.1). In its multiple cloning site region, the T-DNA fragment containing the insect resistance expression cassette (comprising a full-length Cry1Ab/Vip3Da fusion protein expression cassette) and the glyphosate tolerance expression cassette (comprising a C10evo EPSPS protein expression cassette) is inserted. The insect resistance expression cassette contains the Cry1Ab-GAGGAGGG-Vip3Da fusion gene. The fusion gene is driven by the promoter from Turnip vein clearing virus (pCsVMV promoter) and terminated by the NOS terminator from Agrobacterium. The glyphosate tolerance expression cassette is driven by the 35S promoter derived from Cauliflower Mosaic Virus (CaMV), which drives the expression of G10evo EPSPS protein fused with a signal peptide encoding the CTP gene from Arabidopsis at the N-terminus. The termination signal is the 35S gene terminator from CaMV.

The plasmid vector pCAL contains specific components within the T-DNA region (SEQ ID NO:9, also referred to as SEQ ID NO:10, 444-8957bp). The composition and positions of these components are as follows:
1. Right Border (RB) interval sequence: 444-674 (231bp)
2. pCsVMV promoter: 675-1362 (688bp)
3. Interval sequence: 1363-1372 (10bp)
4. Cry1Ab/Vip3Da gene: 1373-5722 (4350bp)
5. Interval sequence: 5723-5728 (6bp)
6. NOS terminator: 5729-5976 (248bp)
7. Interval sequence: 5977-6219 (243bp)
8. pCaMV35S promoter: 6220-7018 (799bp)
9. Interval sequence: 7019-7155 (137bp)
10. CTP gene: 7156-7389 (234bp)
11. g10evo-epsps gene: 7390-8706 (1317bp)
12. Interval sequence: 8707-8718 (12bp)
13. CaMV 35S terminator: 8719-8908 (190bp)
14. Left Border (LB) interval sequence: 8909-8957 (49bp)

TABLE 1
Genome and Genetic Elements Contained in SEQ ID NO: 10

| Position | (Length) | | Name | Function |
|---|---|---|---|---|
| 1-443 | | | Right Flanking Soybean | Soybean genomic sequence flanking |
| | | | Genome Sequence | the T-DNA right border |
| 444-674 | (231 | bp) | RB (T-DNA Right Border) | Interval sequence of the right border |
| | | | Interval Sequence | region where T-DNA is inserted into |
| | | | | the plant genome |
| 675-1362 | (688 | bp) | pCsVMV Promoter | Promoter for the expression of insect |
| | | | | resistance genes |
| 1363-1372 | (10 | bp) | Interval Sequence | Interval sequence used for vector |
| | | | | construction |
| 1373-5722 | (4350 | bp) | cry1Ab/vip3Da | Insect resistance gene |
| 5723-5728 | (6 | bp) | Interval Sequence | Interval sequence used for vector |
| | | | | construction |
| 5729-5976 | (248 | bp) | NOS Terminator | Transcription termination signal |
| 5977-6219 | (243 | bp) | Interval Sequence | Interval sequence used for vector |
| | | | | construction |
| 6220-7018 | (799 | bp) | pCaMV35S Promoter | Promoter for the expression of |
| | | | | glyphosate resistance gene |
| 7019-7155 | (137 | bp) | Interval Sequence | Interval sequence used for vector |
| | | | | construction |
| 7156-7389 | (234 | bp) | CTP | Arabidopsis EPSPS chloroplast signal |
| | | | | peptide |
| 7390-8706 | (1317 | bp) | g10evo-epsps | Confers glyphosate resistance |
| 8707-8718 | (12 | bp) | Interval Sequence | Interval sequence used for vector |
| | | | | construction |
| 8719-8908 | (190 | bp) | CaMV 35S Terminator | Terminator for the expression of |
| | | | | glyphosate resistance |
| 8909-8957 | (49 | bp) | LB (T-DNA Left Border) | Interval sequence of the left border |
| | | | Interval Sequence | region where T-DNA is inserted into |
| | | | | the plant genome |
| 8958-9559 | (602 | bp) | Left Flanking Soybean | Soybean genomic sequence flanking |
| | | | Genome Sequence | the T-DNA left border |

### (2) Agrobacterium Transformation

The plasmid vector pCAL obtained in step (1) is introduced into Agrobacterium strain LBA4404 using electroporation (at 2500V). This process results in the Agrobacterium carrying the transformed vector T-DNA.

(3) Soybean Genetic Transformation

The method for soybean transformation follows the protocol described by Li Guilan et al, in their publication “Research on Agrobacterium-mediated Genetic Transformation of Soybean Cotyledonary Nodes” (Crop Journal, 2005, 31(2): 170-176). The selection compound used in this method is glyphosate. The specific steps are as follows:
\textcircled{1} Soybean seed sterilization (chlorine gas sterilization method): Select mature soybean seeds of cultivar Tianlong 1, which are plump, free from disease spots, cracks, and hardness. Place approximately 150 seeds in a 90*15 mm culture dish, arranged in a single layer. Before sterilization, open the culture dish on a laminar flow hood, turn on the light, and let it blow for 1 hour. Then transfer the culture dish to a desiccator. Place a 250 ml beaker inside the desiccator and add 30 ml of sodium hypochlorite and 70 ml of water to the beaker, mix well. Add 8 ml of concentrated hydrochloric acid (36% mass concentration) to the mixture and immediately cover the desiccator. After approximately 12 hours, open the desiccator and transfer the sterilized soybean seeds to the laminar flow hood for 1 hour of blowing to remove residual chlorine gas.
\textcircled{2} Soybean seed germination: After sterilization, place the soybean seeds, with the hilum facing downwards, into germination medium (GM) so that approximately half of the seeds are submerged in the medium. Place 15 seeds per dish and incubate them under light at 24° C., for 12 hours. This will allow the soy bean seeds to swell.
\textcircled{3} Preparation of Agrobacterium culture: Inoculate the Agrobacterium carrying the transformed vector (prepared in step 1) onto YEP solid medium and incubate it in the dark at 24° C. for 12 hours. Using a sterilized inoculation loop, transfer 1 ml of the Agrobacterium culture onto GADT liquid medium. Vortex the culture and add an appropriate amount of GADT liquid medium to adjust the optical density at 650 nm (OD650) to 0.5, thus obtaining the Agrobacterium culture.
\textcircled{4} Preparation of explants and Agrobacterium inoculation: Place the swollen soybean seeds obtained in step \textcircled{2} on sterilized filter paper. Using a #11 surgical blade, make a slanted cut to remove the tip of the embryonic root from the seed, and then bisect the soybean seed along the axis. Remove the seed coat from the half containing the embryo. Under a dissecting microscope, separate the two cotyledons at the embryonic shoot, exposing the underlying meristem. Gently damage the meristem with the surgical blade to obtain the explant. Immerse the prepared explants in the Agrobacterium culture from step \textcircled{3} for approximately 1.5 hours.
\textcircled{5} Co-cultivation: Discard the excess Agrobacterium culture from step \textcircled{4} and place the explants, with the adhered Agrobacterium, in a culture dish. Place 15 explants per dish and incubate them in the dark at 26° C. for 3 days.
\textcircled{6} Recovery culture: After co-cultivation in step \textcircled{5}, insert the explants at a 30° angle into the recovery medium (RM) with half of the cotyledons submerged in the medium. Place 7 explants per dish. Culture the dishes under conditions of 26° C., 16/8 light/dark cycle, and a light intensity of 3000 lux for 1 week.
\textcircled{7} Induction of adventitious shoots: Transfer the explants from step \textcircled{6} to shoot induction medium (SIM) using the same method. Culture the explants at 26° C., under a 16-hour light/8-hour dark photoperiod, and a light intensity of 3000 lux for 3 weeks.
\textcircled{8} Shoot elongation: Place the explants with developed adventitious shoots on sterilized filter paper. Remove the cotyledons and any yellowed parts. Transfer the adventitious shoots to shoot elongation medium (SEM) with the base submerged in the medium, placing 4-5 explants per dish. Culture the dishes at 26° C., under a 16-hour light/8-hour dark photoperiod, and a light intensity of 3000 lux. Change the medium every 2 weeks until the shoots reach a length of approximately 3 cm.
\textcircled{9} Rooting of shoots: Cut off the shoots from step \textcircled{8} and immerse the cut ends in indole-3-butytric acid (IBA) solution for 2 minutes. Transfer the shoots to rooting medium and continue cultivation at 26° C., under a 16-hour light/8-hour dark photoperiod, and a light intensity of 3000 lux. After 1-2 weeks, when the shoots have developed roots of approximately 2 cm in length, the rooted plantlets are obtained.
\textcircled{10} Transplantation of plantlets: Remove the rooted plantlets from the culture medium and rinse off any residual medium from the roots using tap water. In total, 685 T0 generation transgenic events (independent transformed plants) were generated through plant transformation.

### Example 2: Screening of Transgenic Soybean Events

After hardening, 685 T0 generation transgenic events obtained from Example 1 were transplanted into natural soil in a greenhouse. Among them, 546 seedlings successfully survived in the greenhouse. When the T0 transgenic soybeans reached the vegetative growth stage, they were sprayed with a glyphosate herbicide (effective dosage of 60 g/acre). Among the transgenic events, 87 showed no herbicide damage, 327 exhibited herbicide damage, and 132 events resulted in plant death (Table 2).

TABLE 2
Glyphosate Tolerance of T0 Generation
Soybean Transgenic Events

| Glyphosate Dosage | Herbicide Response | Number of Events |
|---|---|---|
| 60 g/acre | No damage | 87 |
| | Growth stunted, dwarfed | 327 |
| | plants | |
| | Plant death | 132 |

In order to evaluate the copy number of the inserted T-DNA, a quantitative PCR (qPCR) analysis is performed on the 87 transgenic events that showed no herbicide damage. The purpose is to identify and exclude events with two or more copies of the inserted gene. Plant genomic DNA is extracted from the selected transgenic plants using the CTAB method. SYBR Green fluorescence-based qPCR is employed for quantification. The Lectin gene from soybean is chosen as the reference gene. A random transgenic event is selected as the calibrator, and the relative gene content is calculated based on the initial reaction using the Ct value comparison method.

For this experiment, the SYBR Green qPCR assay kit from BIO RAD is used, and the reactions are performed in a Bio-Rad CFX96™ Real-Time PCR system. The reaction conditions and procedures follow the instructions provided with the SYBR Green qPCR assay kit. The primer sequences used for amplification are as follows:

TABLE 3
Primer Sequences for Quantitative PCR

| | Primer | |
|---|---|---|
| Sequence ID | Name | Primer Sequence 5′-3′ |
| SEQ ID NO: 11 | qLEC-FS | GCCCTCTACTCCACCCCCAT |
| SEQ ID NO: 12 | qLEC-RS | GCCCATCTGCAAGCCTTTTT |
| SEQ ID NO: 13 | CAL-F | AGCTGGACGCCATCAACA |
| SEQ ID NO: 14 | CAL-R | CTCAGGTACTCCACCTGCA |

Based on the analysis of G10 gene copy numbers, it has been confirmed that the exogenous gene has integrated into the chromosomal genome of the tested soybean plants. Among them, there are 54 transgenic soybean events with a single copy integration.

The selected descendants from these 54 single-copy transgenic events were subjected to a higher dose of glyphosate tolerance testing. They were sprayed with a glyphosate herbicide at a dosage of 120 g/acre. The results revealed that there are 5 transgenic events showing tolerance to the higher concentration of glyphosate. These events are named CAL16, CAL29, CAL13, CAL39, and CAL56.

TABLE 4
Glyphosate Tolerence of T1 Generation
Soybean Transgenic Events

| Glyphosate Dosage | Herbicide Response | Number of Events |
|---|---|---|
| 120 g/acre | No damage | 5 |
| | Growth Stunted, Dwarfed | 35 |
| | plants | |
| | Plant death | 13 |

Five transformed events, CAL16, CAL29, CAL13, CAL39, and CAL56, were selected for bioassay against cotton bollworm.

Bioassay method: 1% agar was sterilized under high temperature and pressure and allowed to cool slightly. Then, 1 ml of the agar was added to each well of a Corning Costar 24-well plate. After the agar solidified, soybean leaves were punched with a 10 mm diameter puncher and placed flat in the wells of the 24-well plate, with one plate assigned to each transformed event. Cotton bollworm (Helicoverpa armigera) neonate larvae were individually transferred to each well using a brush, with one larva per well. After transferring the larvae, the 24-well plate was covered with a lid and sealed with 3M microporous breathable tape. The plate was then placed in a growth chamber at 28° C., 70% humidity, with a light cycle of 16 hours of light and 8 hours of darkness. After three days, the feeding behavior and mortality rate were recorded by photographing. Under the same conditions, non-transgenic soybean cultivar Tianlong No.1 was used as a control.

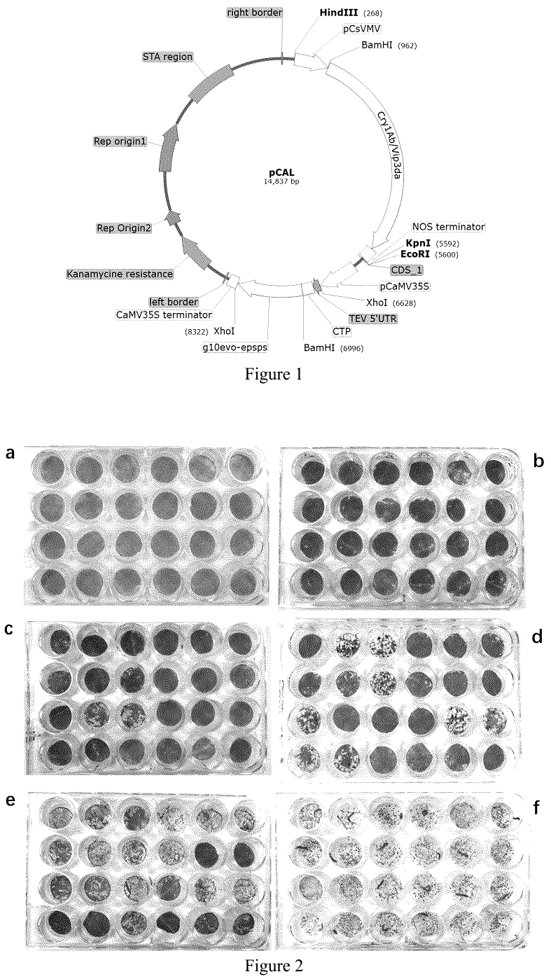

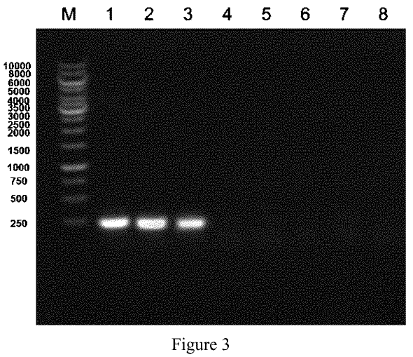

The results, as shown in FIG. 2, indicate that CAL16 had minimal feeding by cotton bollworms. Although CAL29 and CAL13 had a few wells with feeding, upon inspection at the end of the bioassay, all the larvae in those wells had died. CAL39 had a relatively higher number of wells with feeding, and during the third-day inspection, a few wells still had surviving cotton bollworms. However, after five days, all the larvae in those wells had died. CAL56 experienced severe feeding, with only ¼ of the wells having dead cotton bollworms during the third-day inspection, while the rest of the wells showed varying degrees of feeding. The non-transgenic control cultivar, Tianlong No.1, was heavily consumed by cotton bollworms and was almost entirely consumed within three days.

Based on the assessment of insect resistance, glyphosate tolerance, and field performance characteristics, the transformed event CAL16 showed outstanding performance. CAL16 exhibited good resistance to insects and tolerance to glyphosate, with the insertion of a single copy of the exogenous gene. It also demonstrated excellent agronomic traits and stable inheritance of insect resistance and glyphosate tolerance characteristics.

### Example 3: Detection of Soybean Transformed Event CAL16

### (1) Extraction of Soybean Genome

The CTAB (cetyltrimethylammonium bromide) method was used to extract genomic DNA from the soy bean transformed event CAL16.

Take 1000 mg of young leaves from the T0 generation of the soybean transformed event CAL16 and grind them into powder in liquid nitrogen. Add 0.8 mL of preheated CTAB buffer (20 g/L CTAB, 1.4 M NaCl, 100 mM Tris-HCl, 20 mM EDTA, dissolved in water, pH 8.0) to a water bath at 65° C. Thoroughly mix the leaf powder with the buffer and incubate in the water bath at 65° C., for 60 minutes.

Add an equal volume of chloroform, invert and mix well. Centrifuge at 12000 rpm for 10 minutes. Transfer the supernatant to a new centrifuge tube.

Add 0.7 times the volume of isopropanol, gently shake the tube, and centrifuge at 12000 rpm for 1 minute. Collect the DNA pellet at the bottom of the tube. Discard the supernatant. Add 1 mL of 75% ethanol to wash the DNA pellet. Centrifuge at 12000 rpm for 1 minute. Repeat the wash step once. Dry the pellet in a clean bench.

Dissolve the DNA pellet in an appropriate amount of TE buffer (10 mM Tris-HCl, 1 mM EDTA, dissolved in water, pH 8.0). Measure the DNA concentration using Nanodrop and store for further use.

### (2) Analysis of Flanking DNA Sequences

The TAIL-PCR (Thermal Asymmetric Interlaced PCR) method, as reported by Liu et al. (Liu, Plant Journal 1995, 8(3): 457-463), was used to determine the sequence of the regions flanking the insertion point of the exogenous gene in the selected transformed event CAL16, as screened in Example 1. This method involves three nested specific primers combined with degenerate primers in a series of consecutive PCR amplifications, selectively amplifying the target fragments at different annealing temperatures. Three nested specific PCR primers, LB-SP1, LB-SP2, and LB-SP3, were designed based on the left and right border regions of the T-DNA. RB-SP1, RB-SP2, and RB-SP3 were used in PCR amplification with the degenerate primer set AD4L. The primer sequences are provided in Table 5, PCR reaction conditions in Table 6, and PCR reaction system in Table 7.

TABLE 5
TAIL-PCR Primer Sequences

| Primer ID | Primer | Sequence |
|---|---|---|
| SEQ ID NO: 15 | LB-SP1 | TTTCTCCATAATAATGTGTGAGTAGTTCCC |
| SEQ ID NO: 16 | LB-SP2 | CTCATGTGTTGAGCATATAAGAAACCCTTAG |
| SEQ ID NO: 17 | LB-SP3 | CTAAAACCAAAATCCAGTACTAAAATCC |
| SEQ ID NO: 18 | RB-SP1 | CTTGGCACTGGCCGTCGTTT |
| SEQ ID NO: 19 | RB-SP2 | GACTGGGAAAACCCTGGCGTT |
| SEQ ID NO: 20 | RB-SP3 | AGCTGGCGTAATAGCGAAGAGG |
| SEQ ID NO: 21 | AD4L | AGGTTATGCTANTCAGSTWTSGWGWT |

TABLE 6
TAIL-PCR Reaction Conditions

| Reaction | Step | Settings | Cycle Number |
|---|---|---|---|
| First | 1 | 94° C., 2 min; 95° C., 1 min | 1 |
| Round | 2 | 94° C., 1 min; 62° C., | 5 |
| | | 1 min; 72° C., 3 min | |
| | 3 | 94° C., 1 min; 25° C., 3 min; | 1 |
| | | ramping to 72° C., 3 min; | |
| | | 72° C., 2.5 min | |
| | 4 | 94° C., 30 s; 68° C., | |
| | | 1 min; 72° C., 2.5 min | |
| | | 94° C., 30 s; 68° C., | 15 |
| | | 1 min; 72° C., 2.5 min | |
| | | 94° C., 30 s; 44° C., | |
| | | 1 min; 72° C., 2.5 min | |
| | 5 | 72° C., 5 min | 1 |
| Second | 1 | 94° C., 2 min | 1 |
| Round | 2 | 94° C., 30 s; 63° C., | 15 |
| | | 1 min; 72° C., 2 min | |
| | | 94° C., 30 s; 63° C., | |
| | | 1 min; 72° C., 2 min | |
| | | 94° C., 30 s; 44° C., | |
| | | 1 min; 72° C., 2 min | |
| | 3 | 72° C., 5 min | 1 |
| Third | 1 | 94° C., 2 min | 1 |
| Round | 2 | 94° C., 30 s; 63° C., | 15 |
| | | 1 min; 72° C., 2 min | |
| | | 94° C., 30 s; 63° C., | |
| | | 1 min; 72° C., 2 min | |
| | | 94° C., 30 s; 44° C., | |
| | | 1 min; 72° C., 2 min | |
| | 3 | 72° C., 10 min | 1 |

TABLE 7
PCR Reaction System

| Reagent | Volume | |
|---|---|---|
| Template DNA | 1 | μl |
| Forward primer (10 μM) | 2 | μl |
| Reverse primer (10 μM) | 2 | μl |
| 2*Easy Taq PCR SuperMix | 25 | μl |
| ddH₂O | 20 | μl |
| Total volume | 50 | μl |

In the first round of reactions, LB-SP1/RB-SP1 and AD4L primers were used with the CAL16 genomic DNA as the template.

In the second round of reactions, LB-SP2/RB-SP2 and AD4L primers were used with a 1000-fold dilution of the products from the first round as the template.

In the third round of reactions, LB-SP3/RB-SP3 and AD4L primers were used with a 1000-fold dilution of the products from the second round as the template.

The PCR products from the third round were purified using the PCR product recovery kit from Axygen. The purified products were then ligated into the PMD20-T cloning vector (TaKaRa, Code: D107A), followed by transformation into Escherichia coli. The positive clones obtained were sequenced. The obtained sequence information was compared and analyzed with the soybean genome sequence database available online (http://www.soybase.org) to search for similar soybean genomic sequences.

### (3) Integration of CAL16 into the Genome Sequence Information

The flanking sequences upstream and downstream of the confirmed insertion site, as well as the sequences of the exogenous insect resistance gene expression cassette and herbicide resistance gene expression cassette, were assembled to form the transformation event described in this invention. The nucleotide sequence is designated as SEQ ID NO:10, which includes the genomic and genetic elements as listed in Table 1. The soybean transgenic event CAL16, corresponding to the SEQ ID NO:10 sequence, is preserved in the form of soybean (Glycine max) CAL16 seeds at the China Center for Type Culture Collection, with the preservation number CCTCC NO: P202205, and the preservation date is Apr. 18, 2022.

### Example 4: Specific Detection of Transgenic Soybean Event CAL16

This example describes a method for identifying the presence of the CAL16 transgenic soybean event in soybean samples. A pair of PCR primers and a probe are designed to detect the insertion T-DNA sequence of the CAL16 transgenic soybean event and its flanking soybean genomic sequence, which are covered by SEQ ID NO:1-10.

The PCR primers and probe for this example are as follows: SQ111, SQ112, and PB113. The forward primer SQ111 (SEQ ID NO:22) has the same nucleotide sequence as positions 8929 to 8954 of SEQ ID NO:10, positions 999 to 1024 of SEQ ID NO:8, and positions 9 to 34 of SEQ ID NO:6. The reverse primer SQ112 (SEQ ID NO:23) has the same nucleotide sequence as the reverse complement of positions 9042 to 9069 of SEQ ID NO:10 and positions 1112 to 1139 of SEQ ID NO:8. The probe PB113 (SEQ ID NO:24) has the same nucleotide sequence as positions 8996 to 9010 of SEQ ID NO:10, positions 1066 to 1080 of SEQ ID NO:8, and positions 76 to 95 of SEQ ID NO:6. PCR primers SQ111 (SEQ ID NO:22) and SQ112 (SEQ ID NO:23) amplify a unique 141-nucleotide fragment at the correct junction of the CAL16 event's genomic/insert DNA. Probe PB113, when labeled with a fluorescence tag (e.g., 6FAMTM), can be used to detect the PCR products of primers SQ111 and SQ112, thereby identifying the presence of DNA derived from the CAL16 event in the sample.

SEQ ID NO:22:
agtacattaaaaacgtccgcaatgtg;
SEQ ID NO: 23:
tgcaaatattttgttgctacatgttgac;
SEQ ID NO: 24:
cccaggcttatcttt.

Apart from SQ111 (SEQ ID NO:22), SQ112 (SEQ ID NO:23), and PB113 (SEQ ID NO:24), it would be obvious to a person skilled in the art that other primers and/or probes can be designed to amplify and/or hybridize the unique presence of DNA derived from event CAL16 within the sample and for detecting the presence of DNA derived from event CAL16 within the sample.

According to standard molecular biology laboratory practices, PCR assays for event identification have been developed for detecting event CAL16 DNA in samples. The parameters of the standard PCR assay or PCR assay with optimized conditions involve primer sets and probes (i.e., probes labeled with fluorescent tags such as 6FAMTM) specific for detecting the presence of DNA derived from event CAL16 in samples SQ111 (SEQ ID NO:22), SQ112 (SEQ ID NO:23), and PB113 (SEQ ID NO:24). Controls for the PCR reaction include internal control primers and internal control probes (e.g., labeled with VIC\hat{ }TM) specific to a single copy gene in the soybean genome. Those skilled in the art would know how to design primers specific to a single copy gene in the soybean genome. Generally, the optimized parameters for detecting event CAL16 DNA in samples include primer and probe concentrations, template DNA quantity; and PCR amplification cycle parameters.

### Example 5: Identification of any Breeding-active Tissue Containing Transgenic Soybean Event CAL16

This example utilizes a pair of primers to detect any breeding-active tissue containing transgenic soy bean event CAL16 through PCR amplification. The amplification product that confirms the presence of transgenic soybean event CAL16 contains at least 11 consecutive nucleotides in the form of SEQ ID NO:1 or SEQ ID NO:2 or SEQ ID NO:3 or SEQ ID NO:4 or SEQ ID NO:5 or SEQ ID NO:6 or SEQ ID NO:7 or SEQ ID NO:8 or SEQ ID NO:9 or SEQ ID NO:10. The primers used include the primer set based on the flanking sequences and insert of the expression cassette (SEQ ID NO:9).

In this example, to obtain diagnostic amplification products for SEQ ID NO:1, SEQ ID NO:3, or SEQ ID NO:5, a forward primer molecule, SQ114 (SEQ ID NO:25), was designed based on bases 1 to 1610 of SEQ ID NO:7. Additionally, a reverse primer molecule, SQ115 (SEQ ID NO:26), was designed based on bases 1 to 8514 of the insert's expression cassette DNA sequence (position 1 to 8514 of SEQ ID NO:9). These primer molecules have adjacent nucleotides of sufficient length to specifically hybridize to SEQ ID NO:7 and SEQ ID NO:9. The sequence (SEQ ID NO:25) of the forward oligonucleotide primer SQ114 is identical to the nucleotide sequence located at positions 296 to 323 of SEQ ID NO:10 and positions 296 to 323 of SEQ ID NO:7. The sequence (SEQ ID NO:26) of the reverse oligonucleotide primer SQ115 is identical to the reverse complementary nucleotide sequence located at positions 500 to 525 of SEQ ID NO:10, positions 57 to 82 of SEQ ID NO:9, and positions 500 to 525 of SEQ ID NO:7.

SEQ ID NO: 25:
gacaccaataatggtagaatccttggag;
SEQ ID NO: 26:
gcctgaatggcgaatgctagagcagc.

In ths example, DNA was extracted from the leaves, pods, and seeds of the T0 generation plants of transgenic soybean event CAL16 using the CTAB method. The extracted DNA was used as a template for PCR amplification with the primers SQ114 and SQ115, following the reaction system described in Table 8. The amplification products were then subjected to agarose gel electrophoresis, and the results are shown in FIG. 3. Under the same conditions, Tianlong 1 (non-transgenic soybean), Zhonghuang 6106 (transgenic soybean from the Chinese Academy of Agricultural Sciences), conventional rice, and transgenic insect-resistant cotton were used as controls. A blank control without template DNA was also included.

The PCR reaction program was as follows: 3 minutes of denaturation at 95° C., followed by 15 seconds of denaturation at 95° C., 30 seconds of annealing at 58° C., and 30 seconds of extension at 72° C. This cycle was repeated 32 times, followed by a final extension at 72° C., for 3 minutes.

TABLE 8
PCR Reaction System

| | | |
|---|---|---|
| Reagent | Concentration | Final Volume |

| | | | |
|---|---|---|---|
| TaKaRa LA Taq ® | 0.5× | 15 | μL |

| | | | | |
|---|---|---|---|---|
| 10 μmol/L Forward Primer | 0.3 | μmol/L | 1.0 | μL |
| 10 μmol/L Forward Primer | 0.3 | μmol/L | 1.0 | μL |
| 200 mg/L DNA Template | 13.3 | mg/L | 2.0 | μL |

| | | | |
|---|---|---|---|
| ddH₂O | — | 11 | μL |
| Total Volume | — | 30.0 | μL |

According to the electrophoresis results of the amplification products using the SQ114 and SQ115 primers, only the CAL16 sample showed a specific band of approximately 230 bp, which is consistent with the expected size. No specific bands were detected in other samples that do not contain the CAL16 genomic DNA. The primer pair provided in this invention allows for the specific detection of the presence of the CAL16 event (FIG. 3).

In addition to SQ114 (SEQ ID NO:25) and SQ115 (SEQ ID NO:26), it would be evident to those skilled in the art that other primers can be designed to amplify unique sequences within SEQ ID NO:10 for the specific detection of DNA derived from transgenic soybean event CAL16 in the sample. Other primer sequences can be selected by those skilled in the art in the field of DNA amplification methods from SEQ ID NO:7, SEQ ID NO:8, or SEQ ID NO:9. It is within the scope of the present invention to use these DNA primer sequences when the method of this example is modified. The primer sequences capable of obtaining amplification products from samples containing CAL16 include at least one DNA primer sequence derived from SEQ ID NO:7, SEQ ID NO:8, or SEQ ID NO:9.

### Example 6: Protein Expression Level Determination

Different generations (T4, T5, T6) of transgenic soybean event CAL16 were sampled, including leaf samples V4 and V6, root samples R2 and R6, pod sample R6, stem sample R6, and seed sample R8. The protein expression levels of Cry1Ab/Cry1Ac and G10 EPSPS of these samples at different stages were determined using the Cry1Ab/Cry1Ac ELISA kit (EnviroLogix, USA) and the G10 EPSPS enzyme-linked immunosorbent assay kit (Shanghai Biolink Biotechnology Co., Ltd.), respectively. The results are shown in Table 9 and Table 10. The results demonstrate the stable hereditary expression of exogenous proteins in transgenic soybean event CAL16.

Cry1Ab/Cry1Ac ELISA Kit Procedure:
1. Sample and Positive Control Preparation: grind the test samples in liquid nitrogen and weigh 20 mg of the ground sample, add 1 ml of sample extraction buffer to the weighed sample, dilute the positive control provided in the kit with 2 ml of sample extraction buffer, mix thoroughly and let it sit on ice for 3 minutes. Centrifuge at 12,000 g for 10 minutes, take the supernatant and dilute it 20-200 times with PBS for further analysis:
2. Incubation: add 50 μl of Cry1Ab/Cry1Ac enzyme conjugate to each well of the ELISA plate, add 50 μl of the prepared sample and different concentrations of Cry1Ab/Vip3Da proteins (used for the standard curve) to the corresponding sample wells, mix well and seal the ELISA plate with parafilm. Incubate on a horizontal shaker at room temperature, 180 rpm, for 2 hours:
3. Plate Washing: Wash the plate three times with wash buffer. Add 300 μl of wash buffer to each well, fill the plate, and then discard the solution, after washing, invert the plate to remove any remaining liquid:
4. Color Development: add 100 μl of chromogenic substrate to each well. Mix well and incubate at room temperature for 15-20 minutes:
5. Stop Reaction: add 100 μl of stop buffer to each well. Mix thoroughly and allow it to stand for 30 minutes:
6. Detection: measure the absorbance of different samples at 450 nm using a Thermo MK3 microplate reader, use the absorbance values of the positive control to generate a standard curve for the quantification of the target protein.

G10 EPSPS ELISA Kit Procedure:
1. Sample and Positive Control Preparation: grind the test samples in liquid nitrogen and weigh 20 mg of the ground sample, add 1 ml of sample extraction buffer to the weighed sample, mix thoroughly and let it sit on ice for 3 minutes, centrifuge at 12,000 g for 10 minutes, take the supernatant and dilute it 200-1000 times with dilution buffer for further analysis:
2. Sample Incubation: add the diluted samples and different concentrations of G10evo positive protein (used for the standard curve) to the ELISA plate, 100 μl per well, incubate on a horizontal shaker at room temperature, 180 rpm, for 45 minutes:
3. Plate Washing: wash the plate three times with wash buffer, add 300 μl of wash buffer to each well, fill the plate, and then discard the solution, after washing, invert the plate and tap it gently to remove any remaining liquid:
4. Enzyme-Linked Antibody Incubation: add 100 μl of enzyme-linked antibody to each well, incubate on a horizontal shaker at room temperature, 180 rpm, for 30 minutes.
5. Plate Washing: wash the plate three times with wash buffer, add 300 μl of wash buffer to each well, fill the plate, and then discard the solution, after washing, invert the plate and tap it gently to remove any remaining liquid:
6. Color Development: add 100 μl of chromogenic substrate to each well, incubate at room temperature for 15-20 minutes:
7. Stop Reaction: add 100 μl of stop buffer to each well, mix thoroughly and allow it to stand for 30 minutes:
8. Detection: measure the absorbance of different samples at 450 nm using a Thermo MK3 microplate reader, use the absorbance values of the positive control to generate a standard curve for the quantification of the target protein.

TABLE 9
Protein Expression levels of Cry1Ab/Vip3Da in Different
Generations of Transgenic Soybean Event

| | Generation T₄ | Generation T₅ | Generation T₆ |
|---|---|---|---|
| | μg/g fwt ± SD* | μg/g fwt ± SD | μg/g fwt ± SD |
| Soybean | (Measurement | (Measurement | (Measurement |
| Tissue | Range) | Range) | Range) |
| Leaf V4 | 23.5 ± 3.6 | 23.8 ± 4.1 | 22.4 ± 3.3 |
| | (17.8-29.1) | (18.7-29.4) | (18.3-27.0) |
| Leaf V6 | 22.8 ± 3.2 | 22.3 ± 3.6 | 22.5 ± 3.6 |
| | (18.9-27.2) | (17.5-27.3) | (17.6-27.6) |
| Leaf R2 | 23.6 ± 2.4 | 24.0 ± 3.4 | 23.8 ± 3.9 |
| | (19.6-27.3) | (20.1-28.5) | (18.7-28.4) |
| Leaf R6 | 23.7 ± 2.3 | 23.8. ± 3.4 | 23.1 ± 3.7 |
| | (20.5-26.7) | (18.5-27.7) | (19.1-27.5) |
| Pod R6 | 11.6 ± 1.7 | 11.4 ± 1.5 | 11.5 ± 1.7 |
| | (9.7-13.9) | (9.1-13.2) | (8.5-13.6) |
| Stem R6 | 11.2 ± 2.2 | 11.2 ± 2.1 | 11.2 ± 2.0 |
| | (8.1-13.7) | (8.7-13.8) | (7.9-13.9) |
| Root R6 | 5.9 ± 0.8 | 5.5 ± 1.3 | 5.6 ± 1.4 |
| | (4.6-6.9) | (4.1-7.2) | (4.1-7.9) |
| Seed R8 | 16.7 ± 3.6 | 16.2. ± 3.6 | 16.7 ± 3.9 |
| | (12.5-20.3) | (11.3-21.1) | (10.8-20.7) |

*Protein expression levels are reported in micrograms per gram fresh weight(μg/g fw). The values represent the mean ± standard deviation. n = 20 samples. The range in parentheses indicates the maximum and minimum values of the ELISA measurements.

TABLE 10
Protein Expression levels of G10evo-EPSPS in Different
Generations of Transgenic Soybean Event

| | Generation T₄ | Generation T₅ | Generation T₆ |
|---|---|---|---|
| | μg/g fwt ± SD* | μg/g fwt ± SD | μg/g fwt ± SD |
| Soybean | (Measurement | (Measurement | (Measurement |
| Tissue | Range) | Range) | Range) |
| Leaf V4 | 36.7 ± 13.0 | 36.6 ± 11.3 | 36.5 ± 10.7 |
| | (20.4-54.7) | (23.1-52.4) | (22.4-47.9) |
| Leaf V6 | 44.1 ± 14.1 | 44.3 ± 15.0 | 43.6 ± 12.9 |
| | (25.3-59.7) | (26.9-59.9) | (27.6-60.4) |
| Leaf R2 | 46.1 ± 11.9 | 44.6 ± 11.5 | 43.7 ± 11.5 |
| | (31.7-61.5) | (32.5-59.1) | (29.5-57.3) |
| Leaf R6 | 48.3 ± 11.3 | 48.5 ± 12.8 | 48.6 ± 12.7 |
| | (31.6-60.7) | (34.6-64.9) | (31.9-65.7) |
| Pod R6 | 17.6 ± 5.4 | 16.1 ± 5.7 | 16.7 ± 5.4 |
| | (9.6-23.7) | (9.1-24.4) | (8.3-21.5) |
| Stem R6 | 91.7 ± 14.5 | 88.5 ± 14.3 | 91.6 ± 12.7 |
| | (72.3-108.6) | (68.9-104.7) | (72.6-105.8) |
| Root R6 | 22.3 ± 8.3 | 22.5 ± 8.8 | 22.1 ± 8.9 |
| | (10.7-30.6) | (10.4-31.9) | (9.9-32.0) |
| Seed R8 | 116.8 ± 19.3 | 117.6 ± 18.6 | 116.5 ± 21.3 |
| | (91.9-133.4) | (93.7-135.8) | (91.6-143.7) |

*Protein expression levels are reported in micrograms per gram fresh weight(μg/g fw). The values represent the mean ± standard deviation. n = 20 samples. The range in parentheses indicates the maximum and minimum values of the ELISA measurements.

### Example 7: Insect Resistance Assessment of Transgenic Soybean Event CAL16

### 1. Laboratory Bioassay

In this experiment, the T4, T5, and T6 generations of transgenic soybean event CAL16, as well as the non-transgenic parent soybean variety Tianlong-1, were tested for their insect resistance against the fall armyworm (Spodoptera frugiperda), beet armyworm (Spodoptera exigua), cotton bollworm (Helicoverpa armigera), and corn earworm (Helicoverpa zea) through indoor bioassays. V4 stage leaves were collected from different generations of CAL16 and the parent variety Tianlong-1 and brought back to the laboratory. Ten newly hatched larvae of each insect species were placed on the leaves of each tested soybean sample. The experiment was replicated 10 times for each generation and insect species. Mortality rates were recorded at 24 hours, 48 hours, and 72 hours after infestation. The results are shown in Table 11. The results indicate that all larvae died within 2-3 days after infestation on transgenic soybean event CAL16.

TABLE 11
Insect resistance of Different Generations of CAL16 Soybeans
at V4 Stage against Four Target Insects(Laboratory Bioassay)

| | | 24 h | 48 h | 72 h |
|---|---|---|---|---|
| Target | | Mortality | Mortality | Mortality |
| Insect | Genetype | (%) | (%) | (%) |
| Fall | CAL16 T₄ | 27.5 ± 9.4 a | 96.3 ± 6.8 a | 100.0 ± 0.0 a |
| Armyworm | CAL16 T₅ | 34.4 ± 6.7 a | 88.2 ± 8.3 a | 100.0 ± 0.0 a |
| | CAL16 T₆ | 29.4 ± 7.2 a | 96.3 ± 7.1 a | 100.0 ± 0.0 a |
| | CK | 9.1 ± 7.1 b | 14.9 ± 6.7 b | 19.6 ± 5.7 b |
| Beet | CAL16 T₄ | 25.3 ± 6.5 a | 88.7 ± 7.3 a | 100.0 ± 0.0 a |
| Armyworm | CAL16 T₅ | 23.6 ± 7.6 a | 86.6 ± 8.1 a | 100.0 ± 0.0 a |
| | CAL16 T₆ | 22.5 ± 8.5 a | 88.5 ± 6.7 a | 100.0 ± 0.0 a |
| | CK | 10.2 ± 7.6 b | 13.1 ± 7.5 b | 15.2 ± 7.6 b |
| Cotton | CAL16 T₄ | 24.3 ± 7.0 a | 79.6 ± 9.6 a | 100.0 ± 0.0 a |
| Bollworm | CAL16 T₅ | 25.2 ± 8.5 a | 64.2 ± 9.8 a | 100.0 ± 0.0 a |
| | CAL16 T₆ | 23.1 ± 7.9 a | 71.3 ± 8.7 a | 100.0 ± 0.0 a |
| | CK | 24.7 ± 7.0 a | 35.8 ± 9.5 b | 41.5 ± 7.3 b |
| Corn | CAL16 T₄ | 10.3 ± 9.5 b | 100.0 ± 0.0 a | 100.0 ± 0.0 a |
| Earworm | CAL16 T₅ | 20.4 ± 8.7 a | 100.0 ± 0.0 a | 100.0 ± 0.0 a |
| | CAL16 T₆ | 20.2 ± 8.4 a | 100.0 ± 0.0 a | 100.0 ± 0.0 a |
| | CK | 9.7 ± 7.5 b | 12.3 ± 7.9 b | 14.3 ± 8.1 b |

Note:
In Table 11, a and b indicate significant differences between means.

### 2. Field Performance Testing of Insect Resistance

In this experiment, the T4, T5, and T6 generations of transgenic soybean event CAL16 were tested for their insect resistance against the fall armyworm (Spodoptera frugiperda), beet armyworm (Spodoptera exigua), cotton bollworm (Helicoverpa armigera), and corn earworm (Helicoverpa zea) in field conditions. Forty-eight plots with dimensions of 5 m×5 m were set up in the field, with a spacing of 1 m between plots. CAL16 soybeans and non-transgenic soybean variety Tianlong-1 were planted in separate plots using a double-seed method, with a spacing of 25 cm between plants and 50 cm between rows. The experiment was replicated three times for each soybean variety. Ten plants were selected from each plot, and ten first-instar larvae were placed on each plant. After 14 days, the damage caused by insect pests was assessed, and the insect resistance was classified. The results in Table 12 show that CAL16 soybeans exhibited high resistance to the tested insect pests.

The resistance levels were classified based on a 9-level standard (Marcon et al., 1999). Levels 1-3 represent pinhole-like injuries (1: rare, dispersed; 2: moderate quantity; 3: large quantity). Levels 4-6 represent injuries the size of a matchstick head (4: rare, dispersed; 5: moderate quantity; 6: large quantity). Levels 7-9 represent injuries larger than a matchstick head (7: rare, dispersed; 8: moderate quantity; 9: large quantity). Resistance level classification: Levels 1-2 (high resistance), levels 3-4 (resistant), levels 5-6 (susceptible), levels 7-9 (highly susceptible).

TABLE 12
Insect resistance of Different Generations of CAL16 Soybeans
at V4 Stage against Four Target Insects(Field Bioassay)

| Insect type | Genetype | Resistance Level | Resistance type |
|---|---|---|---|
| Fall | CAL16 T₄ | 1.0 | Highly resistant |
| Armyworm | CAL16 T₅ | 1.0 | Highly resistant |
| | CAL16 T₆ | 1.0 | Highly resistant |
| | CK | 8.6 | Highly susceptible |
| Beet | CAL16 T₄ | 1.0 | Highly resistant |
| Armyworm | CAL16 T₅ | 1.0 | Highly resistant |
| | CAL16 T₆ | 1.0 | Highly resistant |
| | CK | 8.5 | Highly susceptible |
| Cotton | CAL16 T₄ | 1.0 | Highly resistant |
| Bollworm | CAL16 T₅ | 1.0 | Highly resistant |
| | CAL16 T₆ | 1.0 | Highly resistant |
| | CK | 8.3 | Highly susceptible |
| Corn | CAL16 T₄ | 1.0 | Highly resistant |
| Earworm | CAL16 T₅ | 1.0 | Highly resistant |
| | CAL16 T₆ | 1.0 | Highly resistant |
| | CK | 8.4 | Highly susceptible |

### Example 8: Transgenic Soybean Event CAL16 Glyphosate Tolerance Test

A randomized complete block design was used in this experiment, with a total of 24 plots. Each plot had an area of 5 m×5 m and was divided into two groups: one for planting transgenic soybean event CAL16 and the other for planting non-transgenic soybean variety Tianlong-1. The spacing between plants was 25 cm, and the row spacing was 50 cm. There was a 1 m gap between each plot. Each type of soybean was replicated three times. During the V3 growth stage, the following steps were taken: 1) No herbicide application; 2) Application of a medium dose of glyphosate at an effective rate of 60 grams per acre; 3) Application of a medium dose of glyphosate at twice the effective rate, 120 grams per acre; 4) Application of a medium dose of glyphosate at four times the effective rate, 240 grams per acre. One week, two weeks, and four weeks after the herbicide application, the following parameters were recorded: emergence rate, plant height (measured on the five tallest plants), and herbicide injury symptoms (evaluated on the five plants with the mildest symptoms). The grading of herbicide injury symptoms was conducted according to the standard GB/T 17980.42-2000. The formula for calculating the herbicide damage rate is as follows:

$$X \% = \frac{Σ ( N \times S )}{T \times M} \times 100$$

(X-herbicide damage rate in percentage; N-number of plants with the same level of herbicide injury; S-injury level; T-total number of plants evaluated; M-highest level).

To compare the differences in emergence rate, seedling rate, and herbicide damage rate between different treatments of transgenic soybean event CAL16 and non-transgenic soybean variety Tianlong-1, you can use the analysis of variance (ANOVA) method. The results are presented in Table 13, showing the field test results of glyphosate on transgenic soybean event CAL16, indicating a high level of tolerance to glyphosate.

TABLE 13
Glyphosate tolerance of soybean event CAL16

| | | | After 1 Week | | After 2 Week | | After 4 Week | |
|---|---|---|---|---|---|---|---|---|
| | | | | Herbicide | | Herbicide | | Herbicide |
| Treatment | | Seedling | | damage | | damage | | damage |
| (Effective | | Rate(%) | Height | rate | Height | rate | Height | rate |
| Does) | Material | % | (cm) | (%) | (cm) | (%) | (cm) | (%) |
| Untreated | Tianlong-1 | 100 | 14.5 ± 1.3 | — | 26.5 ± 2.1 | — | 38.1 ± 3.3 | — |
| | CAL16 | 100 | 14.6 ± 1.5 | — | 27.1 ± 2.2 | — | 38.2 ± 3.5 | — |
| 60 | Tianlong-1 | 0 | — | 100 | — | 100 | — | 100 |
| g/acre | CAL16 | 100 | 14.3 ± 2.1 | 0 | 26.4 ± 2.3 | 0 | 38.1 ± 3.2 | 0 |
| 120 | Tianlong-1 | 0 | — | 100 | — | 100 | — | 100 |
| g/acre | CAL16 | 100 | 13.9 ± 1.9 | 0 | 26.4 ± 2.5 | 0 | 38.2 ± 3.6 | 0 |
| 240 | Tianlong-1 | 0 | — | 100 | — | 100 | — | 100 |
| g/acre | CAL16 | 100 | 13.8 ± 1.5 | 0 | 26.2 ± 1.9 | 0 | 38.1 ± 3.5 | 0 |

### Example 9: Hybridization to Generate Offspring Containing Transgenic Soybean Event CAL16

To produce soybean plants or plant parts with enhanced agronomic, insect-resistant, or herbicide-tolerant traits, hybridization can be performed between soybean plants containing the transgenic soybean event CAL16 and potential soybean plants containing any other soybean event or their combinations. The phenotypes of the offspring plants are evaluated to determine the characteristics obtained.

The specific steps for hybridization are as follows:
1. Flower selection: Choose flowers from healthy, undamaged, and vigorously growing non-transgenic soybean plants containing the CAL16 event. Select flowers that have petals just starting to emerge from the sepals and where the color of the petals is visible.
2. Emasculation: Hold the flower stem and the base of the flower with the thumb and index finger of the left hand. Use a pair of forceps in the right hand to gently remove most of the sepals by pulling them downward or at an angle. This exposes the fused petals. At this point, hold the upper part of the fused petals (about one-third from the flag petal) at a slight angle (approximately 45 degrees) towards the keel petal (to avoid damaging the stigma) and gently pull it upward in a slanting motion towards the flag petal.
3. Pollination: Tear off the sepals at the keel petal position of the CAL16 transgenic soybean parent flower. Separate the fused petals between the keel petals, exposing the yellowish anthers (appearing fluffy on the surface). Using forceps, gently extract the entire pollen mass from the middle part of the stamen. With the left hand, lightly squeeze the male flower, align the anthers with the stigma, and gently rub them together a few times to transfer the pollen. Then, carefully insert the pollen mass onto the stigma, ensuring it is inserted upside down to both continue the dispersal of pollen and protect the exposed stigma.

Hybridization can be used to introduce various enhanced traits into the offspring plants generated through plant breeding using the transgenic soybean event CAL16. These traits can extend beyond lepidopteran resistance and glyphosate resistance of the CAL16 event and may include, but are not limited to, above-ground insect control, herbicide tolerance, nematode resistance, drought tolerance, virus resistance, enhanced fungal control, bacterial resistance, male sterility, cold tolerance, salt tolerance, increased yield, enhanced oil composition, increased oil content, enhanced nutrient use efficiency, or altered amino acid content. Examples of transgenic events with improved agronomic traits are well-known in the field.

The following is a non-exhaustive list of possible transgenic soybean lines that can be used for breeding with the transgenic soybean event CAL16 to confer enhanced traits in soybean plants, plant parts, seeds, or commercial products. Breeding can involve any one or a combination of the following: herbicide tolerance: soybean GTS 40-3-2, MON87708, MON89788, A2704-12, A2704-21, A5547-35, A5547-127, BPS-CV127-9, DP356043, GU262, W62, W98, DAS-44406-6, DAS-68416-4, FG72, BPS-CV127-9, SYHT04R, SYHTOH2, 3560.4.3.5, EE-GM3, pDAB4472-1606, pDAB4468-0416, pDAB8291.45.36.127, AAD-12; insect resistance: MON87701, DAS-81419-2; enhanced oil composition: DP-305423, G94-1, G94-19, G168, OT96-15, MON87705, MON87769; increased yield: MON 87712.

1. A transgenic soybean event CAL16, characterized by the DNA molecule obtained by inserting exogenous genes into the soybean genome between the 3′ end shown in SEQ ID NO:27 and the 5′ end shown in SEQ ID NO:28 on chromosome 18; The exogenous genes include a glyphosate resistance gene cassette and an insect resistance gene cassette.

2. A transgenic soybean event CAL16, as claimed in claim 1, characterized by the glyphosate resistance gene cassette that comprises the following elements: a promoter, pCaMV35S promoter, used for the expression of the glyphosate gene g10evo-epsps; an EPSPS chloroplast transit peptide derived from Arabidopsis; the g10evo-epsps gene itself; and the terminator, CaMV 35S terminator; the insect resistance gene cassette comprises the following elements: the pCsVMV promoter; the cry1Abg/Vip3Da fusion gene, which confers insect resistance; and the NOS terminator.

3. A transgenic soybean event CAL16, as claimed in claim 1, characterized by the following: the nucleotide sequence of the DNA molecule of the transgenic soybean event CAL16 is as shown in SEQ ID NO:10.

4. A method for detecting the nucleotide sequence of the transgenic soybean event CAL16 as claimed in claim 1, characterized by the following: the nucleotide sequence for detection includes SEQ ID NO:1 or its complementary sequence, and/or SEQ ID NO:2 or its complementary sequence.

5. The nucleotide sequence as described in claim 4, characterized by the following: the nucleotide sequence includes one of SEQ ID NO:3, SEQ ID NO:4, SEQ ID NO:5, SEQ ID NO:6, SEQ ID NO:7, SEQ ID NO:8, SEQ ID NO:9, or SEQ ID NO:10, or their complementary sequences.

6. A method for detecting the presence of the DNA molecules of the transgenic soybean event CAL16 as claimed in claim 1 in a sample, characterized by the following steps: (1) Contacting the sample to be tested with a DNA probe or primer pair in a nucleic acid amplification reaction solution; The primer pair includes a first primer and a second primer. The first primer is either SEQ ID NO:23 or SEQ ID NO:25; the second primer is either SEQ ID NO:22 or SEQ ID NO:26. The DNA probe is as shown in SEQ ID NO:24; (2) Performing a nucleic acid amplification reaction; (3) Detecting the presence of amplification products; The amplification products include at least 11 consecutive nucleotides from one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, or their complementary sequences.

7. A method for cultivating insect-resistant soybean plants containing the transgenic soybean event CAL16 as claimed in claim 1 is characterized by the following steps: planting soybean seeds containing specific nucleotide sequences, harvesting soybean plants with significantly enhanced resistance to Lepidopteran insects compared to non-transgenic soybean plants, thereby protecting the soybean plants from insect infestation; the specific nucleotide sequences are selected from one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, or their complementary sequences; the Lepidopteran insects include but are not limited to the Fall Armyworm, Beet Armyworm, Pink Bollworm, and Soybean Looper.

8. A method for cultivating herbicide-tolerant soybean plants containing the transgenic soybean event CAL16 as claimed in claim 1 is characterized by the following steps: planting soybean seeds containing specific nucleotide sequences, applying herbicide treatment, harvesting soybean plants with significantly enhanced herbicide tolerance compared to non-transgenic soybean plants; the specific nucleotide sequences are selected from one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, or their complementary sequences; the herbicide described includes glyphosate.

9. A method for controlling field weeds in soybean plants of the transgenic soybean event CAL16, as claimed in claim 1, comprising: planting transgenic soybean plants containing a specific nucleic acid sequence in the region; applying an effective dose of glyphosate herbicide to kill weeds; wherein the transgenic soybean genome contains a specific nucleic acid sequence from the transgenic soybean event CAL16, and the specific nucleic acid sequence comprises one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10, or their complementary sequences.

10. A method for generating soybean plants with insect resistance and/or glyphosate tolerance, comprising: hybridizing soybean plants containing a specific nucleic acid sequence in the region with another soybean plant to produce offspring plants; harvesting plants that exhibit significantly improved tolerance to herbicides and/or insect resistance compared to other plants lacking the specific nucleic acid sequence; wherein the specific nucleic acid sequence is derived from the transgenic soybean event CAL16, and the specific region of the nucleic acid sequence comprises one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10, or their complementary sequences.

11. A method for producing transgenic plant cells derived from the transgenic soybean event CAL16 as claimed in claim 1, characterized by the following: the transgenic plant cells are obtained by introducing the specific nucleic acid sequence from the transgenic soybean event CAL16 into the plant genome, and the specific region of the nucleic acid sequence comprises one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10, or their complementary sequences.

12. A method for producing commodities or agricultural products derived from the transgenic soybean event CAL16 as claimed in claim 1, characterized by the following: the soybean commodities or agricultural products comprise: soybean oil, soybean protein, soybean meal, soybean flour, soybean flakes, soybean skin, soy milk, soy cheese, soybean wine, animal feed containing soybean, paper containing soybean, cheese containing soybean, soybean biomass, and fuel products produced using soybean plants or parts of soybean plants.


SEQUENCE LISTING (28 sequences)

SEQ ID NO: 1 - DNA, 25 bp, synthetic construct
  source 1..25: mol_type = other DNA
tcaacatatctcaaacactgatagt

SEQ ID NO: 2 - DNA, 25 bp, synthetic construct
  source 1..25: mol_type = other DNA
ttaagttgtccactattattgtttt

SEQ ID NO: 3 - DNA, 60 bp, synthetic construct
  source 1..60: mol_type = other DNA
aagtttctcttctactcctcatgtcaacatatctcaaacactgatagtttaaactgaagg

SEQ ID NO: 4 - DNA, 60 bp, synthetic construct
  source 1..60: mol_type = other DNA
acgtccgcaatgtgttattaagttgtccactattattgtttttgtattatattttcccag

SEQ ID NO: 5 - DNA, 100 bp, synthetic construct
  source 1..100: mol_type = other DNA
ttcctcttttactttctgttaagtttctcttctactcctcatgtcaacatatctcaaacactgatagtttaaactgaaggcgggaaacgacaatctgatc

SEQ ID NO: 6 - DNA, 100 bp, synthetic construct
  source 1..100: mol_type = other DNA
gttaattcagtacattaaaaacgtccgcaatgtgttattaagttgtccactattattgtttttgtattatattttcccaggcttatcttttatcaatgag

SEQ ID NO: 7 - DNA, 1610 bp, synthetic construct
  source 1..1610: mol_type = other DNA
tttggcaaagggagaagggaatgaaaaagatgaatagcacaagttttcaaggtttggaaaaaccaaaaaactttggaaagcttttagcaaaaggaaggagaagaagaagaagaagaagaagaagttcaaagagattcaaggcttgtaaaggattgtataagattgattggaaaagtgtattgaaaagcaaatcatgtcaacattaatgagcctgggatgctctaaacacatgattggagatgcatcaaagctcactcatatttctcccaagaatagtggacatgtgacttatggcgacaccaataatggtagaatccttggagttggaaaaactcttgtgtgtttgtctccttcttccctactcttttactttccattgtgcattttaatttcctcttttactttctgttaagtttctcttctactcctcatgtcaacatatctcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttccagaaggtaattatccaagatgtagcatcaagaatccaatgtttacgggaaaaactatggaagtattatgtgaactcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcactagtgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtggatccaacaatggacaacaaccccaacatcaacgagtgcatcccctacaactgcctgagcaaccccgaggtggaggtgctgggcggcgagcgcatcgagaccggctacacccccatcgacatcagcctgagcctgacccagttcctgctgagcgagttcgtgcccggcgccggcttcgtgctgggcctggtggacatcatctggggcatcttcggccccagccagtgggacgccttcctggtgcaga

SEQ ID NO: 8 - DNA, 1639 bp, synthetic construct
  source 1..1639: mol_type = other DNA
atgttcctcggtcctcttcttcctgacggactcgaacttagactcaccggtgatatcaagtcccacgctcctcttagacagacacttgacaccctctctgatttcggtgttagagctactgcctccgatgaccttagaagaatctccatccctggtggtcagaagtacagaccaggtagagtgctcgttcctggtgattaccctggttccgctgctatccttaccgccgctgctcttctcccaggtgaggttagactttctaaccttagagaacacgacctccagggtgagaaggaagctgtgaacgttcttagagagatgggtgctgatatcgttagagaaggtgatacccttaccgtgagaggtggtagacctctccacgctgttactagagatggtgattccttcaccgacgccgtgcaagctcttaccgctgctgctgccttcgctgagggtgataccacctgggaaaacgttgctactcttagactcaaggaatgcgatagaatctctgacaccagagctgagcttgaaagacttggtcttagagcaagagagaccgccgattctctctccgttactggttctgctcaccttgctggtggtatcaccgctgatggtcacggtgaccacagaatgatcatgcttctcacccttcttggtctcagagcagatgctccacttagaatcaccggtgcacaccacatcagaaagtcctaccctcagttcttcgctcaccttgaagctcttggtgctagattcgaatacgctgaggctaccgcctaataggagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaatccagtacattaaaaacgtccgcaatgtgttattaagttgtccactattattgtttttgtattatattttcccaggcttatcttttatcaatgagttatatataaagaaataatcagtcaacatgtagcaacaaaatatttgcagtaataataataacgttaaacaatagaaattaaaaaaacctaacaacaaatgtcttgattttaaagacttgtgttcacatgatcatttgatcaagtaaaagataacttcctaaggcaagaatgaaagtagtgagatgtttttagttttttttttccacacaaactaattctaaaaacattttatcttaaacaaaaatacaattaacaaagaaaatatttttcaacaatctcatactaatgtaaaacttcaggaaatacataatataataaaaacatttttaatggaacatcatgcattgtgttttcatccattaatttttcatgcttactagaaaggaacttaatcatatccattatagatattttgatacaacatcatgctaccgcagaaaagactatgcaagaagaaagtataaaacatttttctctcttaagactgttggaaaataaaacaaaaatgaaggaaaataaatacgaagaagatgcacagtcttgaatta

SEQ ID NO: 9 - DNA, 8514 bp, synthetic construct
  source 1..8514: mol_type = other DNA
tcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttccagaaggtaattatccaagatgtagcatcaagaatccaatgtttacgggaaaaactatggaagtattatgtgaactcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcactagtgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtggatccaacaatggacaacaaccccaacatcaacgagtgcatcccctacaactgcctgagcaaccccgaggtggaggtgctgggcggcgagcgcatcgagaccggctacacccccatcgacatcagcctgagcctgacccagttcctgctgagcgagttcgtgcccggcgccggcttcgtgctgggcctggtggacatcatctggggcatcttcggccccagccagtgggacgccttcctggtgcagatcgagcagctgatcaaccagcgcatcgaggagttcgcccgcaaccaggccatcagccgcctggagggcctgagcaacctgtaccaaatctacgccgagagcttccgcgagtgggaggccgaccccaccaaccccgccctgcgcgaggagatgcgcatccagttcaacgacatgaacagcgccctgaccaccgccatccccctgttcgccgtgcagaactaccaggtgcccctgctgagcgtgtacgtgcaggccgccaacctgcacctgagcgtgctgcgcgacgtcagcgtgttcggccagcgctggggcttcgacgccgccaccatcaacagccgctacaacgacctgacccgcctgatcggcaactacaccgaccacgccgtgcgctggtacaacaccggcctggagcgcgtgtggggtcccgacagccgcgactggatcaggtacaaccagttccgccgcgagctgaccctgaccgtgctggacatcgtgagcctgttccccaactacgacagccgcacctaccccatccgcaccgtgagccagctgacccgcgagatttacaccaaccccgtgctggagaacttcgacggcagcttccgcggcagcgcccagggcatcgagggcagcatccgcagcccccacctgatggacatcctgaacagcatcaccatctacaccgacgcccaccgcggcgagtactactggagcggccaccagatcatggccagccccgtcggcttcagcggccccgagttcaccttccccctgtacggcaccatgggcaacgctgcacctcagcagcgcatagtggcacagctgggccagggagtgtaccgcaccctgagcagcaccctgtaccgtcgacctttcaacatcggcatcaacaaccagcagctgagcgtgctggacggcaccgagttcgcctacggcaccagcagcaacctgcccagcgccgtgtaccgcaagagcggcaccgtggacagcctggacgagatcccccctcagaacaacaacgtgccacctcgacagggcttcagccaccgtctgagccacgtgagcatgttccgcagtggcttcagcaacagcagcgtgagcatcatccgtgcacctatgttcagctggattcaccgcagtgccgagttcaacaacatcatccccagcagccagatcacccagatccccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgaacgagctgttcaccagcagcaaccagatcggcctgaagaccgacgtgaccgactaccacatcgatcaggtgcgaggccccggtggagcaggtggagcaggtggagggggtaaaggaaacaacaccaagctgaacgccagggccctgccgagcttcatcgactacttcaacggcatctacggcttcgccaccggcatcaaggacatcatgaacatgatcttcaagaccgacaccggcggcaacctgaccctggacgagatcctgaagaaccagcagctgctgaacgagatcagcggcaagctggacggcgtgaacggcagcctgaacgacctgatcgcccagggcaacctgaacaccgagctgagcaaggagatcctgaagatcgccaacgagcagaaccaggtgctgaacgacgtgaacaacaagctggacgccatcaacaccatgctgcacatctacctgccgaagatcaccagcatgctgagcgacgtgatgaagcagaactacgccctgagcctgcaggtggagtacctgagcaagcagctgaaggagatcagcgacaagctggacgtgatcaacgtgaacgtgctgatcaacagcaccctgaccgagatcaccccggcctaccagaggatcaagtacgtgaacgagaagttcgaggagctgaccttcgccaccgagaccaccctgaaggtgaagaaggacagcagcccggccgacatcctggacgagctgaccgagctgaccgagctggccaagagcgtgaccaagaacgacgtggacggcttcgagttctacctgaacaccttccacgacgtgatggtgggcaacaacctgttcggcaggagcgccctgaagaccgccagcgagctgatcgccaaggagaacgtgaagaccagcggcagcgaggtgggcaacgtgtacaacttcctgatcgtgctgaccgccctgcaggccaaggccttcctgaccctgaccacctgcaggaagctgctgggcctggccgacatcgactacaccagcatcatgaacgagcacctgaacaaggagaaggaggagttcagggtgaacatcctgccgaccctgagcaacaccttcagcaacccgaactacgccaaggtgaagggcagcgacgaggacgccaagatgatcgtggaggccaagccgggccacgccctggtgggcttcgagatcagcaacgacagcatgaccgtgctgaaggtgtacgaggccaagctgaagcagaactaccaggtggacaaggacagcctgagcgaggtgatctacagcgacatggacaagctgctgtgcccggaccagagcgagcagatctactacaccaacaacatcgtgttcccgaacgagtacgtgatcaccaagatcgacttcaccaagaagatgaagaccctgaggtacgaggtgaccgccaacagctacgacagcagcaccggcgagatcgacctgaacaagaagaaggtggagagcagcgaggccgagtacaggaccctgagcgccaacaacgacggcgtgtacatgccgctgggcgtgatcagcgagaccttcctgaccccgatcaacggcttcggcctgcaggccgacgagaacagcaggctgatcaccctgacctgcaagagctacctgagggagctgctgctggccaccgacctgagcaacaaggagaccaagctgatcgtgccgccgatcagcttcatcagcaacatcgtggagaacggcaacctggagggcgagaacctggagccgtggatcgccaacaacaagaacgcctacgtggaccacaccggcggcatcaacggcaccaaggtgctgtacgtgcacaaggacggcgagttcagccagttcgtgggcggcaagctgaagagcaagaccgagtacgtgatccagtacatcgtgaagggcaaggccagcatctacctgaaggacgagaacaccggctacatccactacgaggacaccaacaacaacctggaggactaccagaccatcaacaagaggttcaccaccggcaccgacctgaagggcgtgtacctgatcctgaagagccagaacggcgacgaggcctggggcgacaacttcatcatcctggagatcagcccgagcgagaagctgctgagcccggagctgatcaacaccaacaactggaccagcaccggcagcaccaacatcagcggcaacaccctgaccctgtaccagggcggcaggggcatcctgaagcagaacctgcagctggacagcttcagcacctacagggtgtacttcagcgtgagcggcgacgccaacgtgaggatcaggaacagcagggaggtgctgttcgagaagaggtacatgagcggcgccaaggacgtgagcgagatgttcaccaccaagttcgagaaggacaacttctacatcgagctgagccagggcaacaacctgtacggcggcccgatcgtgcacttctacgacgtgagcatcaagtaagagctcttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtcaacacaacatatacaaaacaaacgaatctcaagcaatcaagcattctacttctattgcagcaatttaaatcatttcttttaaagcaaaagcaattttctgaaaattttcaccatttacgaacgatagccatggctcaagttagcagaatctgcaatggtgtgcagaacccatctcttatctccaatctctctaaatccagtcaaaggaaatctcccttatcggtttctctgaagactcagcagcatccacgagcttatccaatttcttcatcttggggattgaagaagagtgggatgactttaattggctctgagcttcgtcctcttaaggtcatgtcttctgtttccacggcggagaagggatccgacgctcttccagctaccttcgacgttatcgtgcatccagctagagaactcagaggtgaacttagagcacagccatccaagaactacaccactagatacctcctcgccgctgctctcgctgagggtgaaaccagagttgttggtgtggctacctctgaggatgccgaagctatgctcagatgcctcagagattggggtgctggtgttgagcttgttggtgatgacgccgtgatcagaggtttcggtgctagaccacaggctggtgttacccttaacccaggtaacgctgctgcggtggccagactccttatgggtgttgctgctctcacctctggtacaactttcgttaccgattaccctgattcccttggtaagagacctcagggtgaccttcttgaagccctcgaaagacttggtgcttgggtgtcctccaacgatggtagactccctatctccgtttccggtccagttagaggtggtacagtggaggtttccgccgaaagatcctcccagtacgcttccgcccttatgttcctcggtcctcttcttcctgacggactcgaacttagactcaccggtgatatcaagtcccacgctcctcttagacagacacttgacaccctctctgatttcggtgttagagctactgcctccgatgaccttagaagaatctccatccctggtggtcagaagtacagaccaggtagagtgctcgttcctggtgattaccctggttccgctgctatccttaccgccgctgctcttctcccaggtgaggttagactttctaaccttagagaacacgacctccagggtgagaaggaagctgtgaacgttcttagagagatgggtgctgatatcgttagagaaggtgatacccttaccgtgagaggtggtagacctctccacgctgttactagagatggtgattccttcaccgacgccgtgcaagctcttaccgctgctgctgccttcgctgagggtgataccacctgggaaaacgttgctactcttagactcaaggaatgcgatagaatctctgacaccagagctgagcttgaaagacttggtcttagagcaagagagaccgccgattctctctccgttactggttctgctcaccttgctggtggtatcaccgctgatggtcacggtgaccacagaatgatcatgcttctcacccttcttggtctcagagcagatgctccacttagaatcaccggtgcacaccacatcagaaagtcctaccctcagttcttcgctcaccttgaagctcttggtgctagattcgaatacgctgaggctaccgcctaataggagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaatccagtacattaaaaacgtccgcaatgtgttattaagttgtc

SEQ ID NO: 10 - DNA, 9559 bp, synthetic construct
  source 1..9559: mol_type = other DNA
tttggcaaagggagaagggaatgaaaaagatgaatagcacaagttttcaaggtttggaaaaaccaaaaaactttggaaagcttttagcaaaaggaaggagaagaagaagaagaagaagaagaagttcaaagagattcaaggcttgtaaaggattgtataagattgattggaaaagtgtattgaaaagcaaatcatgtcaacattaatgagcctgggatgctctaaacacatgattggagatgcatcaaagctcactcatatttctcccaagaatagtggacatgtgacttatggcgacaccaataatggtagaatccttggagttggaaaaactcttgtgtgtttgtctccttcttccctactcttttactttccattgtgcattttaatttcctcttttactttctgttaagtttctcttctactcctcatgtcaacatatctcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttccagaaggtaattatccaagatgtagcatcaagaatccaatgtttacgggaaaaactatggaagtattatgtgaactcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcactagtgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtggatccaacaatggacaacaaccccaacatcaacgagtgcatcccctacaactgcctgagcaaccccgaggtggaggtgctgggcggcgagcgcatcgagaccggctacacccccatcgacatcagcctgagcctgacccagttcctgctgagcgagttcgtgcccggcgccggcttcgtgctgggcctggtggacatcatctggggcatcttcggccccagccagtgggacgccttcctggtgcagatcgagcagctgatcaaccagcgcatcgaggagttcgcccgcaaccaggccatcagccgcctggagggcctgagcaacctgtaccaaatctacgccgagagcttccgcgagtgggaggccgaccccaccaaccccgccctgcgcgaggagatgcgcatccagttcaacgacatgaacagcgccctgaccaccgccatccccctgttcgccgtgcagaactaccaggtgcccctgctgagcgtgtacgtgcaggccgccaacctgcacctgagcgtgctgcgcgacgtcagcgtgttcggccagcgctggggcttcgacgccgccaccatcaacagccgctacaacgacctgacccgcctgatcggcaactacaccgaccacgccgtgcgctggtacaacaccggcctggagcgcgtgtggggtcccgacagccgcgactggatcaggtacaaccagttccgccgcgagctgaccctgaccgtgctggacatcgtgagcctgttccccaactacgacagccgcacctaccccatccgcaccgtgagccagctgacccgcgagatttacaccaaccccgtgctggagaacttcgacggcagcttccgcggcagcgcccagggcatcgagggcagcatccgcagcccccacctgatggacatcctgaacagcatcaccatctacaccgacgcccaccgcggcgagtactactggagcggccaccagatcatggccagccccgtcggcttcagcggccccgagttcaccttccccctgtacggcaccatgggcaacgctgcacctcagcagcgcatagtggcacagctgggccagggagtgtaccgcaccctgagcagcaccctgtaccgtcgacctttcaacatcggcatcaacaaccagcagctgagcgtgctggacggcaccgagttcgcctacggcaccagcagcaacctgcccagcgccgtgtaccgcaagagcggcaccgtggacagcctggacgagatcccccctcagaacaacaacgtgccacctcgacagggcttcagccaccgtctgagccacgtgagcatgttccgcagtggcttcagcaacagcagcgtgagcatcatccgtgcacctatgttcagctggattcaccgcagtgccgagttcaacaacatcatccccagcagccagatcacccagatccccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgaacgagctgttcaccagcagcaaccagatcggcctgaagaccgacgtgaccgactaccacatcgatcaggtgcgaggccccggtggagcaggtggagcaggtggagggggtaaaggaaacaacaccaagctgaacgccagggccctgccgagcttcatcgactacttcaacggcatctacggcttcgccaccggcatcaaggacatcatgaacatgatcttcaagaccgacaccggcggcaacctgaccctggacgagatcctgaagaaccagcagctgctgaacgagatcagcggcaagctggacggcgtgaacggcagcctgaacgacctgatcgcccagggcaacctgaacaccgagctgagcaaggagatcctgaagatcgccaacgagcagaaccaggtgctgaacgacgtgaacaacaagctggacgccatcaacaccatgctgcacatctacctgccgaagatcaccagcatgctgagcgacgtgatgaagcagaactacgccctgagcctgcaggtggagtacctgagcaagcagctgaaggagatcagcgacaagctggacgtgatcaacgtgaacgtgctgatcaacagcaccctgaccgagatcaccccggcctaccagaggatcaagtacgtgaacgagaagttcgaggagctgaccttcgccaccgagaccaccctgaaggtgaagaaggacagcagcccggccgacatcctggacgagctgaccgagctgaccgagctggccaagagcgtgaccaagaacgacgtggacggcttcgagttctacctgaacaccttccacgacgtgatggtgggcaacaacctgttcggcaggagcgccctgaagaccgccagcgagctgatcgccaaggagaacgtgaagaccagcggcagcgaggtgggcaacgtgtacaacttcctgatcgtgctgaccgccctgcaggccaaggccttcctgaccctgaccacctgcaggaagctgctgggcctggccgacatcgactacaccagcatcatgaacgagcacctgaacaaggagaaggaggagttcagggtgaacatcctgccgaccctgagcaacaccttcagcaacccgaactacgccaaggtgaagggcagcgacgaggacgccaagatgatcgtggaggccaagccgggccacgccctggtgggcttcgagatcagcaacgacagcatgaccgtgctgaaggtgtacgaggccaagctgaagcagaactaccaggtggacaaggacagcctgagcgaggtgatctacagcgacatggacaagctgctgtgcccggaccagagcgagcagatctactacaccaacaacatcgtgttcccgaacgagtacgtgatcaccaagatcgacttcaccaagaagatgaagaccctgaggtacgaggtgaccgccaacagctacgacagcagcaccggcgagatcgacctgaacaagaagaaggtggagagcagcgaggccgagtacaggaccctgagcgccaacaacgacggcgtgtacatgccgctgggcgtgatcagcgagaccttcctgaccccgatcaacggcttcggcctgcaggccgacgagaacagcaggctgatcaccctgacctgcaagagctacctgagggagctgctgctggccaccgacctgagcaacaaggagaccaagctgatcgtgccgccgatcagcttcatcagcaacatcgtggagaacggcaacctggagggcgagaacctggagccgtggatcgccaacaacaagaacgcctacgtggaccacaccggcggcatcaacggcaccaaggtgctgtacgtgcacaaggacggcgagttcagccagttcgtgggcggcaagctgaagagcaagaccgagtacgtgatccagtacatcgtgaagggcaaggccagcatctacctgaaggacgagaacaccggctacatccactacgaggacaccaacaacaacctggaggactaccagaccatcaacaagaggttcaccaccggcaccgacctgaagggcgtgtacctgatcctgaagagccagaacggcgacgaggcctggggcgacaacttcatcatcctggagatcagcccgagcgagaagctgctgagcccggagctgatcaacaccaacaactggaccagcaccggcagcaccaacatcagcggcaacaccctgaccctgtaccagggcggcaggggcatcctgaagcagaacctgcagctggacagcttcagcacctacagggtgtacttcagcgtgagcggcgacgccaacgtgaggatcaggaacagcagggaggtgctgttcgagaagaggtacatgagcggcgccaaggacgtgagcgagatgttcaccaccaagttcgagaaggacaacttctacatcgagctgagccagggcaacaacctgtacggcggcccgatcgtgcacttctacgacgtgagcatcaagtaagagctcttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtcaacacaacatatacaaaacaaacgaatctcaagcaatcaagcattctacttctattgcagcaatttaaatcatttcttttaaagcaaaagcaattttctgaaaattttcaccatttacgaacgatagccatggctcaagttagcagaatctgcaatggtgtgcagaacccatctcttatctccaatctctctaaatccagtcaaaggaaatctcccttatcggtttctctgaagactcagcagcatccacgagcttatccaatttcttcatcttggggattgaagaagagtgggatgactttaattggctctgagcttcgtcctcttaaggtcatgtcttctgtttccacggcggagaagggatccgacgctcttccagctaccttcgacgttatcgtgcatccagctagagaactcagaggtgaacttagagcacagccatccaagaactacaccactagatacctcctcgccgctgctctcgctgagggtgaaaccagagttgttggtgtggctacctctgaggatgccgaagctatgctcagatgcctcagagattggggtgctggtgttgagcttgttggtgatgacgccgtgatcagaggtttcggtgctagaccacaggctggtgttacccttaacccaggtaacgctgctgcggtggccagactccttatgggtgttgctgctctcacctctggtacaactttcgttaccgattaccctgattcccttggtaagagacctcagggtgaccttcttgaagccctcgaaagacttggtgcttgggtgtcctccaacgatggtagactccctatctccgtttccggtccagttagaggtggtacagtggaggtttccgccgaaagatcctcccagtacgcttccgcccttatgttcctcggtcctcttcttcctgacggactcgaacttagactcaccggtgatatcaagtcccacgctcctcttagacagacacttgacaccctctctgatttcggtgttagagctactgcctccgatgaccttagaagaatctccatccctggtggtcagaagtacagaccaggtagagtgctcgttcctggtgattaccctggttccgctgctatccttaccgccgctgctcttctcccaggtgaggttagactttctaaccttagagaacacgacctccagggtgagaaggaagctgtgaacgttcttagagagatgggtgctgatatcgttagagaaggtgatacccttaccgtgagaggtggtagacctctccacgctgttactagagatggtgattccttcaccgacgccgtgcaagctcttaccgctgctgctgccttcgctgagggtgataccacctgggaaaacgttgctactcttagactcaaggaatgcgatagaatctctgacaccagagctgagcttgaaagacttggtcttagagcaagagagaccgccgattctctctccgttactggttctgctcaccttgctggtggtatcaccgctgatggtcacggtgaccacagaatgatcatgcttctcacccttcttggtctcagagcagatgctccacttagaatcaccggtgcacaccacatcagaaagtcctaccctcagttcttcgctcaccttgaagctcttggtgctagattcgaatacgctgaggctaccgcctaataggagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaatccagtacattaaaaacgtccgcaatgtgttattaagttgtccactattattgtttttgtattatattttcccaggcttatcttttatcaatgagttatatataaagaaataatcagtcaacatgtagcaacaaaatatttgcagtaataataataacgttaaacaatagaaattaaaaaaacctaacaacaaatgtcttgattttaaagacttgtgttcacatgatcatttgatcaagtaaaagataacttcctaaggcaagaatgaaagtagtgagatgtttttagttttttttttccacacaaactaattctaaaaacattttatcttaaacaaaaatacaattaacaaagaaaatatttttcaacaatctcatactaatgtaaaacttcaggaaatacataatataataaaaacatttttaatggaacatcatgcattgtgttttcatccattaatttttcatgcttactagaaaggaacttaatcatatccattatagatattttgatacaacatcatgctaccgcagaaaagactatgcaagaagaaagtataaaacatttttctctcttaagactgttggaaaataaaacaaaaatgaaggaaaataaatacgaagaagatgcacagtcttgaatta

SEQ ID NO: 11 - DNA, 20 bp, synthetic construct
  source 1..20: mol_type = other DNA
gccctctactccacccccat

SEQ ID NO: 12 - DNA, 20 bp, synthetic construct
  source 1..20: mol_type = other DNA
gcccatctgcaagccttttt

SEQ ID NO: 13 - DNA, 18 bp, synthetic construct
  source 1..18: mol_type = other DNA
agctggacgccatcaaca

SEQ ID NO: 14 - DNA, 19 bp, synthetic construct
  source 1..19: mol_type = other DNA
ctcaggtactccacctgca

SEQ ID NO: 15 - DNA, 30 bp, synthetic construct
  source 1..30: mol_type = other DNA
tttctccataataatgtgtgagtagttccc

SEQ ID NO: 16 - DNA, 31 bp, synthetic construct
  source 1..31: mol_type = other DNA
ctcatgtgttgagcatataagaaacccttag

SEQ ID NO: 17 - DNA, 28 bp, synthetic construct
  source 1..28: mol_type = other DNA
ctaaaaccaaaatccagtactaaaatcc

SEQ ID NO: 18 - DNA, 20 bp, synthetic construct
  source 1..20: mol_type = other DNA
cttggcactggccgtcgttt

SEQ ID NO: 19 - DNA, 21 bp, synthetic construct
  source 1..21: mol_type = other DNA
gactgggaaaaccctggcgtt

SEQ ID NO: 20 - DNA, 22 bp, synthetic construct
  source 1..22: mol_type = other DNA
agctggcgtaatagcgaagagg

SEQ ID NO: 21 - DNA, 26 bp, synthetic construct
  source 1..26: mol_type = other DNA
aggttatgctantcagstwtsgwgwt

SEQ ID NO: 22 - DNA, 26 bp, synthetic construct
  source 1..26: mol_type = other DNA
agtacattaaaaacgtccgcaatgtg

SEQ ID NO: 23 - DNA, 28 bp, synthetic construct
  source 1..28: mol_type = other DNA
tgcaaatattttgttgctacatgttgac

SEQ ID NO: 24 - DNA, 15 bp, synthetic construct
  source 1..15: mol_type = other DNA
cccaggcttatcttt

SEQ ID NO: 25 - DNA, 28 bp, synthetic construct
  source 1..28: mol_type = other DNA
gacaccaataatggtagaatccttggag

SEQ ID NO: 26 - DNA, 26 bp, synthetic construct
  source 1..26: mol_type = other DNA
gcctgaatggcgaatgctagagcagc

SEQ ID NO: 27 - DNA, 443 bp, synthetic construct
  source 1..443: mol_type = other DNA
tttggcaaagggagaagggaatgaaaaagatgaatagcacaagttttcaaggtttggaaaaaccaaaaaactttggaaagcttttagcaaaaggaaggagaagaagaagaagaagaagaagaagttcaaagagattcaaggcttgtaaaggattgtataagattgattggaaaagtgtattgaaaagcaaatcatgtcaacattaatgagcctgggatgctctaaacacatgattggagatgcatcaaagctcactcatatttctcccaagaatagtggacatgtgacttatggcgacaccaataatggtagaatccttggagttggaaaaactcttgtgtgtttgtctccttcttccctactcttttactttccattgtgcattttaatttcctcttttactttctgttaagtttctcttctactcctcatgtcaacatatc

SEQ ID NO: 28 - DNA, 602 bp, synthetic construct
  source 1..602: mol_type = other DNA
cactattattgtttttgtattatattttcccaggcttatcttttatcaatgagttatatataaagaaataatcagtcaacatgtagcaacaaaatatttgcagtaataataataacgttaaacaatagaaattaaaaaaacctaacaacaaatgtcttgattttaaagacttgtgttcacatgatcatttgatcaagtaaaagataacttcctaaggcaagaatgaaagtagtgagatgtttttagttttttttttccacacaaactaattctaaaaacattttatcttaaacaaaaatacaattaacaaagaaaatatttttcaacaatctcatactaatgtaaaacttcaggaaatacataatataataaaaacatttttaatggaacatcatgcattgtgttttcatccattaatttttcatgcttactagaaaggaacttaatcatatccattatagatattttgatacaacatcatgctactgcagaaaagactatgcaagaagaaagtataaaacatttttctctcttaagactgttggaaaataaaacaaaaatgaaggaaaataaatacgaagaagatgcacagtcttgaatta